(19) United States
(12) Patent Application Publication
(10) Pub. No.: US 2012/0143090 A1
(43) Pub. Date: Jun. 7, 2012

(54) Assessment of Spinal Anatomy

(76) Inventors: Ori Hay, Haifa (IL); Israel Hershkovitz, Hod Hasharon (IL)

(21) Appl. No.: 13/390,166

(22) PCT Filed: Aug. 10, 2010
(86) PCT No.: PCT/IL2010/000646

§ 371 Date: Feb. 13, 2012

Related U.S. Application Data
(60) Provisional application No. 61/234,296, filed on Aug. 16, 2009.

(51) Int. Cl. A61B 5/00

(52) U.S. Cl. 600/587

(57) ABSTRACT

A method for modeling a spine, comprising providing at least one image of the spine, extracting a plurality of anatomical elements of the spine from the at least one image, and constructing a model representing the anatomy of the spine using the anatomical elements.

### FIELD OF THE INVENTION

The invention relates to assessment of the anatomy of the spine. Some embodiments of the invention relate to modeling the anatomy or morphology of the spine.

### BACKGROUND OF THE INVENTION

Low back pain (LBP) is a substantial cause of disability among the working population. LBP is a common disease among the work force of the industrial world, where around 60%-90% are typically affected (see, for example, Frymoyer J W. Back pain and sciatica. N Eng J Med 1988; 318:291-300). Low back pain direct costs are estimated at total of $20-120 bn for the USA each year (see, for example, S. Dagenais, J. Caro and S. Haldeman, A systematic review of low back pain cost of illness studies in the United States and internationally, Spine J 8 (2008), pp. 8-20). Therefore identifying risk factors for LBP could be important both to individuals and the working population. Currently, much of the search for potential risk factors concentrates on the relationship between various occupational activities and LBP. Despite the research, the results obtained are inconsistent, and in many cases chronic pain do not relate to well-defined pathological causes where about 85% of chronic back pain is due to nonspecific (idiopathic) or unknown cause (see, for example, Chou R, Qaseem A, et al. Diagnosis and treatment of low back pain: a joint clinical practice guideline from the American College of Physicians and the American Pain Society. Ann Intern Med. 2007; 147(7):478-491).

### SUMMARY OF THE INVENTION

An aspect of the invention relates to modeling the spine and/or parts thereof. In some embodiments of the invention, modeling is based on extraction of anatomical and/or morphological features or elements of the spine and/or parts thereof from imaging modalities and/or other sources, and consequently constructing a model of the spine or parts thereof.

In some embodiments of the invention, modeling comprises determining the spine shape and/or structure. In some embodiments, modeling comprises determining quantitative geometrical relationships between two or more elements (or components) of the spine. In some embodiments, modeling comprises determining and/or evaluating and/or measuring one or more geometrical properties or attributes of the spine and/or of elements thereof or within elements (or components) thereof. In some embodiments, modeling comprises determining and/or quantifying internal and/or intrinsic properties of the spine and/or parts thereof.

In some embodiments of the invention, a model of a spine represents the anatomy of a spine, typically as a representation of the anatomical and/or morphological elements (or features), preferably with quantitative properties of the elements, or between or within the elements. In some embodiments, a model comprises relationships between the elements, optionally in hierarchical arrangement. Optionally or additionally, a model comprises auxiliary information such as demographic data or norm values or margins or standard deviation thereof.

In some embodiments of the invention, a model is formed or constructed, at least partially, as a data structure (or structures), typically implemented in a memory apparatus of a computer or computer system.

Another aspect of the invention relates to determining a condition or conditions of a person's (patient's) spine based on evaluating one or more anatomical parts and/or elements or features of the spine of the person compared to a model of the spine. Typically an evaluation results with one or more values, comprising one or more of dimensional magnitudes, values of geometrical relations between or within parts of the spine, or quantities of constitution of part of part of the spine.

In some embodiments of the invention, in determining a condition of the spine an evaluated result is weighed against or compared with a norm (e.g. normal range) of the respective part or element or feature, for example, patient's spine curvature vs. a normal curvature range, or patient's disc height between given vertebrae vs. a normal height range of the disc.

In some embodiments of the invention, determining a condition or conditions of a person's spine (test spine) comprises comparing between models of two or more spines (reference spines), or parts thereof, and determining or evaluating a difference or a deviation (or a plurality thereof) between the anatomies, such as between respective (corresponding, analogous) elements and/or properties of two or more spines

In some embodiments, one or more of the models used for comparison with a spine model of a patient (a reference spine model) are based on one or more spines which are considered as regular and/or ordinary and/or typical to represent a standard or benchmark spine or model thereof (normal spine). In some embodiments, one or more of the models are based on one or more spines which are derived from anatomical repository, such as image banks or atlases, or from anatomical models (solid three-dimensional models).

In some embodiments, comparing between models of two or more spines comprises one of (a) comparing at least one of property of the person's spine model with a respective property of a reference spine, or (b) comparing at least one element of the person's spine model with a respective element of a reference spine model, or (c) comparing at least one property of at least one element of the person's spine model with a respective property of a respective element of a reference spine model, or (d) comparing at least one relation property of at least two element or parts thereof of the person's spine model with a respective property of respective elements of a reference spine model, or (e) any combination thereof, wherein, optionally, a reference spine is a benchmark spine of an approximation thereof.

In some embodiments of the invention, a determined condition is decided (e.g. judged or estimated) as anomalous, such as having one of irregularity, asymmetry, deformity or any combinations thereof.

Typically, in some embodiments, a determined condition is decided as anomalous if one or more properties deviate beyond a certain and/or determined limit, for example, as compared to property's norm and standard deviation. In some embodiments, an anomalous condition is judged if only the cumulative deviations of a plurality of properties are beyond a limit while individual property or some properties' deviations are sufficiently small or below the limit, where cumulative deviations are, for example, sum of deviations or other derivation or combination of deviations such as average or standard deviation)

In some embodiments, in determining a condition of a patient's spine additional auxiliary information is also considered. For example, clinical data such as illness or anamnesis and/or demographic information such as gender or race or age. For example, in some embodiments, the standard or benchmark spine is based on, or modified according to, or adapted to demographic characteristic and/or clinical status or clinical information of the patient.

In some preferred embodiments, the determined conditions of a person's spine and/or differences relative to model spine are used for clinical assessment and/or diagnosis of a patient spine. For example, with determined anomalous condition the clinical assessment or diagnosis indicate possible cause or likelihood of a patient's back pain or back related health problem. In some embodiments, the nature of a determined anomalous condition indicate possible or potential remedies or treatments.

In some embodiments of the invention, comparing a person's model from different times is used to assess an appearance or remission or progress of a spine's disorder or anomaly.

Another aspect of the invention relates to presentation of a model of a spine or a combined or a combination of a plurality of models and properties thereof, adapted for and/or suitable for aiding in determining an assessment and/or diagnosis by an operator such as a clinician. The presentation comprises graphical entities (e.g. vertebrae) and/or textual values (e.g. distances, densities). Optionally the presentation provides capabilities for modification of a model or properties thereof.

In some embodiments, a patient's spine or part thereof is presented, optionally together with some additional data such clinical information. In some embodiments, a patient spine or part thereof is presented together with a reference spine or part thereof, demonstrating differences or anomaly of the patient's spine with respect to the reference. The presentation is either manually or automatically initiated and is interactively controlled by an operator (e.g. a clinician or surgeon) for convenient viewing and examination.

In preferred embodiments, the presented data or part thereof is modifiable, either graphically or alphanumerically, typically interactively by an operator. For example, smoothing a rugged surface, or fixing a numerical value. In some embodiments, data related to the patient or spine thereof is added or removed. For example, adding or removing annotations on a model or part thereof, providing an assessment or diagnosis, modifying a previous diagnosis or removing an indication of an illness. Optionally, measurements and calculations or other derivations may be performed on or based on the presented data, for example, measurement of areas, volumes or misalignments with a reference.

In preferred embodiments of the invention, methods and procedures for modeling, comparisons of models, assessment or diagnosis, and presentation and modifications as described are represented in a software executable by a computer stored in a computer readable storage medium, and carried out using a computer according to programs stored on or comprised in storage device or devices, optionally aided by additional apparatus such as input and output components or devices.

In the specification and claims the following terms and derivatives and inflections thereof imply the respective non-limiting characterizations below, unless otherwise specified or evident from the context.

Spine—the human vertebral column or backbone or spinal column, a column usually consisting of 24 vertebrae, the sacrum, intervertebral discs, and the coccyx (tailbone) situated in the dorsal aspect of the torso, separated by intervertebral discs and houses the spinal cord in its spinal canal and provides an attachment for the muscles of the trunk. A spine optionally and additionally denotes ligaments and muscles connected thereto. A spine may also imply a corresponding or analogous member in other vertebrates.

Anatomy, morphology (of a spine or parts thereof)—denote and relate to shape, form, structure and constitution of the spine or part or parts thereof, and may be used herein interchangeably.

Property—relates to a value associated (at least approximately) with a part or feature of a spine, or a relation between parts of the spine, or between portions or divisions of a part, such as distance, volume, curvature or density.

Intrinsic (property)—relates to the contents or constitution of an element rather than or in addition to geometrical or shape characteristics.

Elements or features (of a spine or parts thereof)—components and parts that make up the spine, comprising also properties or characteristics of the spine or parts thereof (e.g. metrics, intrinsic properties). Denoted also as ‘elements’ or ‘features’ with respect to a spine.

Model (of a spine or parts thereof)—a quantitative anatomically and/or morphologically interrelated representation of the spine or elements or parts thereof, either as a geometrical representation and/or morphological representation and/or as intrinsic characteristics, optionally accompanied by auxiliary data such as demographic characteristics or clinical data. The representation comprise metrics or properties of parts of the spine as well.

Clinical (data, information)—relates to the patient's health such as present and/or past illnesses (anamnesis), pain or anomalies.

Demographic (data, characteristics, information)—relates to particular features or properties of a patient due to factors such as age, gender or race or combination thereof.

Reference (model)—relates to a spine model with which a patient's spine model is compared.

Norm, normality—relates to dimensional or constitution measures considered or accepted as ordinary and absent of irregularity and malfunction. A norm is usually represented as single value representing the typical measure for the healthy population (e.g. standard deviation and/or a range of values which may vary according to demographic or clinical characteristics of the patient.

Benchmark (spine, model)—relates to a reference spine model and/or elements thereof representing a normal or standard spine and/or elements thereof, optionally also representing and/or adapted to demographic characteristics. Optionally, a benchmark model comprises values indicating normal margins and/or standard deviation of one or more of elements of the spine.

Operator—relates to a person using or operating or controlling an apparatus.

It should be noted that particularly specified solid three-dimensional models are excluded from the term ‘model’ as generally used herein, though solid three-dimensional models may be utilized in forming a model, such as a reference model.

According to an aspect of some embodiments of the present invention there is provided a method for modeling a spine, comprising:

(a) providing at least one image of a spine;

(b) extracting a plurality of anatomical elements of the spine from the at least one image; and

(c) constructing a model representing the anatomy of the spine using the anatomical elements.

In some embodiments, the anatomy of the spine comprises representing quantitative properties of the spine and parts thereof.

In some embodiments, representing the anatomy of the spine comprises representing quantitative morphological and geometrical properties of the spine and parts thereof.

In some embodiments, representing the anatomy of the spine comprises representing quantitative properties of the elements.

In some embodiments, representing the anatomy of the spine comprises representing quantitative geometrical relationships between and within the elements.

In some embodiments, representing the anatomy of the spine comprises representing a hierarchy between the elements.

In some embodiments, the model is constructed as a data structure.

In some embodiments, constructing a model comprises quantitatively determining with respect to the spine or parts thereof at least one of geometrical relationships between a plurality of elements, at least one geometrical property of at least one element, at least one geometrical property within at least one element, or at least one intrinsic property, or any combination thereof.

In some embodiments, the at least one image is provided digitally and the extraction of the elements and construction of the model are carried out by at least one computer according to at least one program comprised in a storage device.

In some embodiments, extracting a plurality of anatomical elements comprises using at least one image processing technique.

In some embodiments, providing at least one image comprises acquiring at least one image from at least one apparatus of CT, MRI, PET, US or X-rays.

In some embodiments, constructing a model comprises constructing a model based on a plurality of images of a plurality of spines.

According to an aspect of some embodiments of the present invention there is provided a method for assessment of a patient's spine, comprising:

(a) providing a model representing the anatomy of the patient's spine by elements of the spine and properties thereof; and

(b) determining a condition of a person's health based on the model of the patient's spine.

In some embodiments, determining a condition comprises evaluating at least one element of the spine, obtaining a result and determining a deviation of the result from a respective norm.

In some embodiments, the norm is provided by a model of a reference spine representing the anatomy of the reference spine by elements of the reference spine and properties thereof.

In some embodiments, a norm is provided as a range of values.

In some embodiments, the norm is based on at least one of a clinical data or demographic characteristic of the patient.

In some embodiments, a patient's spine condition is determined as anomalous if at least one deviation is beyond a limit.

In some embodiments, a patient's spine condition is determined as anomalous if a cumulation of a plurality of deviations is beyond a limit while at least one deviation is below the limit.

In some embodiments, determining a condition comprises:

(a) providing a model of a benchmark spine representing the anatomy of the benchmark spine by elements of the benchmark spine and properties thereof; and

(b) comparing the model of the patient's spine to the model of the benchmark spine.

In some embodiments, the model of the benchmark spine represents at least a part of a normal spine.

In some embodiments, the model of the benchmark spine represents normal margins for at least a part of a spine.

In some embodiments, the model of the benchmark spine is constructed based on elements of a plurality of healthy spines.

In some embodiments, comparing comprises comparing at least one property of the patient's spine model with a respective property of the benchmark spine model.

In some embodiments, comparing comprises comparing at least one element of the patient's spine model with a respective element of the benchmark spine model.

In some embodiments, comparing comprises comparing at least one property of at least one element of the patient's spine model with a respective property of a respective element of the benchmark spine model.

In some embodiments, the model of the benchmark spine is adapted to at least one of demographic characteristic or clinical data of the patient.

In some embodiments, comparing the model of the patient's spine to the model of the benchmark spine comprises determining at least one deviation between respective characteristics of the models.

In some embodiments, a patient's spine condition is determined as anomalous if at least one deviation is beyond a limit.

In some embodiments, a patient's spine condition is determined as anomalous if a cumulation of a plurality of deviations is beyond a limit while at least one deviation is below the limit.

In some embodiments, determining a condition comprises indicating at least one of possible cause or likelihood of a patient's back pain.

In some embodiments, determining a condition comprises indicating a potential remedy.

In some embodiments, the model of a patient's spine is provided as a data structure in a memory device and determining the condition of a person's spine is carried out by at least one medical workstation comprised of at least one computer according to at least one program comprised in a storage device.

In some embodiments, the benchmark model is provided as a data structure in a memory device and comparing the model of the patient's spine to the model of the benchmark spine is carried out by at least one medical workstation comprised of at least one computer according to at least one program comprised in a storage device.

According to an aspect of some embodiments of the present invention there is provided a method for assessment of changes in a patient's spine, comprising:

(a) providing a model representing the anatomy of the patient's spine at one time;

(b) providing a model representing the anatomy of the patient's spine at a second time later than the first time, and;

(c) comparing the model of the second time to the model of the first time.

According to an aspect of some embodiments of the present invention there is provided a method for aiding an assessment of a spine, comprising:

(a) providing a model representing the anatomy of a test spine; and

(b) displaying a presentation of at least a part of the model of the test spine in a manner adapted for determining an assessment of the spine by an operator.

In some embodiments, the presentation comprises at least a graphical presentation, such as an image, volume rendering, or virtual reality.

In some embodiments, the presentation comprises a textual presentation of at least one property relating to the at least a part of the spine.

In some embodiments, the presentation comprises a depiction of the relationship of the at least one part to a respective noun.

In some embodiments, the method further comprises measuring at least one property of the at least a part of the test spine.

In some embodiments, the method further comprises modifying a property of the at least a part of the test spine.

In some embodiments, the method further comprises modifying a graphical representation of the at least a part of the test spine.

In some embodiments, the method further comprises (a) providing a model representing the anatomy of a reference spine, and (b) displaying a presentation of at least a part of the model of the test spine with a presentation of at least a corresponding part of the model of the reference spine wherein the presentation is adapted to portray a difference between the corresponding parts.

In some embodiments, the presentation comprises a depiction of the relationship difference between the corresponding parts to a respective norm.

In some embodiments, the method further comprises measuring the difference between the corresponding parts.

In some embodiments, the presentation is carried out by at least one computer according to at least one program comprised in a storage device.

According to an aspect of some embodiments of the present invention there is provided a computer readable storage medium having data stored therein representing software executable by a computer, the software comprising instructions to at least one of:

(a) extract a plurality of anatomical elements of a spine from an image,

(b) construct a model representing the anatomy of a spine using extracted anatomical elements,

(c) determine a condition of a spine based on a model of a spine,

(d) compare a model of the spine to another model of another spine,

(e) display a presentation of at least a part of a model of a spine in a manner adapted for determining an assessment of the spine by an operator,

(f) display a presentation of at least a part of a model of a spine with a presentation of at least a corresponding part of another model of another spine wherein the presentation is adapted to portray a difference between the corresponding parts, or

(g) any combination of any of (a)-(f).

### BRIEF DESCRIPTION OF THE DRAWINGS

Some non-limiting exemplary embodiments of the invention are illustrated in the following drawings.

Identical or duplicate or equivalent or similar structures, elements, or parts that appear in one or more drawings are generally labeled with the same reference numeral, optionally with an additional letter or letters to distinguish between similar objects or variants of objects, and may not be repeatedly labeled and/or described.

Dimensions of components and features shown in the figures are chosen for convenience or clarity of presentation and are not necessarily shown to scale or true perspective. For convenience or clarity, some elements or structures are not shown or shown only partially and/or with different perspective or from different point of views.

It should be noted that some figures were converted to black-and-white rendering, thereby degrading the pictorial quality such by reducing certain details or texture or fineness.

FIG. 1 schematically illustrates a simplified and abbreviated spine model as a diagram of hierarchies and relations between elements of a spine, as a representation of a more comprehensive spine model, according to exemplary embodiments of the invention;

FIG. 2A illustrates a chart schematically outlining data and actions for assessment of a patient's spine, according to exemplary embodiments of the invention;

FIG. 2B illustrates a chart schematically outlining a further action for assessment of a patient's spine with respect to the chart of FIG. 2A, according to exemplary embodiments of the invention;

FIG. 2C illustrates a chart schematically outlining a further action for assessment of a patient's spine with respect to the chart of FIG. 2B, according to exemplary embodiments of the invention;

FIG. 2D illustrates a chart schematically outlining a further action for assessment of a patient's spine with respect to the chart of FIG. 2C, according to exemplary embodiments of the invention;

FIG. 2E illustrates a chart schematically outlining other data action for assessment of a patient's spine with respect to the chart of FIG. 2D, according to exemplary embodiments of the invention;

FIG. 2F illustrates a chart schematically outlining other data and actions for assessment of a patient's spine with respect to the chart of FIG. 2E, according to exemplary embodiments of the invention;

FIG. 2G illustrates a chart schematically outlining a further action for assessment of a patient's spine with respect to the chart of FIG. 2A, according to exemplary embodiments of the invention;

FIGS. 3A-D schematically illustrate a vertebra from four view sides, with indications on some particular elements;

FIGS. 4A-H schematically illustrates measurements performed on vertebrae or part thereof, according to exemplary embodiments of the invention;

FIG. 5 schematically illustrates how a height of a vertebra, a height of an intervertebral disc, the distance between vertebrae and disc center displacement are measured, according to exemplary embodiments of the invention;

FIG. 6 schematically illustrates how a height of an intervertebral disc is measured, according to exemplary embodiments of the invention;

FIG. 7 schematically illustrates how a Cobb angle is measured, according to exemplary embodiments of the invention;

FIG. 8 schematically illustrates how a sacral slope and a sacral tilt are measured, according to exemplary embodiments of the invention;

FIG. 9A illustrates a two dimensional image of a CT study of a spine, after some pre-processing such as thresholding and region growing, as basis subsequent operations;

FIG. 9B illustrates a spinal canal extraction based on the image of FIG. 9A;

FIG. 9C illustrates detection of vertebrae based on the image of FIG. 9A;

FIG. 9D illustrates identification of vertebrae based on the image of FIG. 9A;

FIG. 9E illustrates segmentation of vertebrae based on the image of FIG. 9A;

FIG. 10A illustrates a lateral view of initial segmentation of the spinal canal;

FIG. 10B illustrates an anterior view of initial segmentation of the spinal canal;

FIG. 10C illustrates a lateral view of the spinal canal after centerline extraction based on an initial segmentation;

FIG. 11 schematically illustrates neighbor vertebra bodies and transverse processes deviations, according to exemplary embodiments of the invention;

FIGS. 12A-C schematically illustrate three views of plurality of spine curves and a representative curve formed as a median of the plurality of curves, according to exemplary embodiments of the invention;

FIGS. 13A-D schematically illustrate test curves deviations from a reference model in sagittal views (13A-B) and coronal views (13C-D), according to exemplary embodiments of the invention;

FIG. 14A illustrates an axial image view of an Average Shape Atlas of abdomen of an individual;

FIG. 14B illustrates the image of FIG. 14A overlaid with probabilistic map of segmented Quadratus Lumborum muscle, according to exemplary embodiments of the invention;

FIG. 15A illustrates a partial view of the image of FIG. 14A, with indications of manual and automatic segmentations, according to exemplary embodiments of the invention;

FIG. 15B illustrates a partial view of the image of FIG. 14A, with indications of probabilistic atlas, according to exemplary embodiments of the invention; and

FIG. 16 schematically illustrates a system comprising components and functional units with some relations therebetween, of according to exemplary embodiments of the invention.

### DESCRIPTION OF EMBODIMENTS OF THE INVENTION

The following description relates to one or more non-limiting examples of embodiments of the invention. The invention is not limited by the described embodiments or drawings, and may be practiced in various manners or configurations or variations. The terminology used herein should not be understood as limiting unless otherwise specified.

The non-limiting section headings used herein are intended for convenience only and should not be construed as limiting the scope of the invention.

### Prerequisites

It is assumed throughout the descriptions and discussions below that the anatomy and morphology of the human (or other vertebrates) spine is known, and therefore is not elaborated herein. Some details and properties of the spine and vertebrae are discussed below as deemed to pertain, without limiting, to the present invention.

### Equipment and Techniques

Throughout the description, images of a spine and/or parts thereof (or of solid three-dimensional models) are obtained by imaging equipment of the art. Optionally, solid three-dimensional models or images from repository (see also below) are measured by measurement tools, such as calipers or co-ordinate measuring machine (CMM) or the corresponding images thereof are measured by using software tools.

Image processing techniques are employed to derive properties and/or representations of the spine or parts thereof.

In typical embodiments, processors and/or computer systems and equipment carry out the computational and processing or the algorithms. For example, personal computers, high performance computers (e.g. mainframes), computers networks, embedded processors, DSP or FPGA or ASIC components, neural networks, expert systems equipment, and possible future computational equipment. In some embodiments fuzzy logic as software tools and/or equipment is employed as well where considered advantageous.

### Imaging (Data Acquisition)

Imaging equipment comprises tools or apparatus such as computerized tomography (CT), magnetic resonance (MRI), Positron Emission Tomography (PET), combine tools such as PET/CT, C-arm multiple X-ray images, or X-rays imaging, optionally employing contrast mediums and employing winnowing (gray scale adjustment) and/or image processing techniques. In some cases images from different modalities are combined such as CT/MRI fusion to provide better quality or combined image of different types of tissues. Optionally, other modalities of the art are used, such as ultrasound (US).

### Image Processing

Image processing algorithm and techniques are known in the art and are progressing as algorithms, software tools and computation equipment are developed.

Image processing techniques comprise, for example and without limiting, edge detection, segmentation (e.g. shape-based segmentation scheme, level sets or active contours segmentation, model fitting, region growing techniques, or morphological segmentation techniques optionally based on features such as image intensities, edges, shape, texture, or combination thereof), clustering, morphological operations (e.g., skeletonization, region growing), thresholding, pattern recognition and matching, feature extraction, texture analysis, correlations (e.g. correlation with images in a depositary), deviations calculations, computational geometry, shortest path using graphs methods or fast marching methods, medial line calculation, watershed methods, distance methods (e.g. Hausdorff distance, mean points distance or median of distances), area and volume determinations, connectivity analysis, Hough transforms, smoothing and sharpening and deblurring (e.g. deconvolution), reconstructions, moments analysis, curve or model fitting (e.g. polynomial fitting), classifications (e.g. statistical analysis, machine learning, neural networks, Gaussian Mixture Models, radial basis function, Support Vector Machines) or rule based methods. See, for some examples, John C. Russ, The Image Processing Handbook, CRC press, ISBN 0849325161, or Bernd Jahne, Practical Handbook on Image Processing for Scientific Applications, CRC press, ISBN 084989062, and other literature of the art.

In some embodiments of the invention, prior information is used in the image processing algorithms or techniques in order to facilitate and operation, obtain faster performance and/or more reliable results. For example, images from an image repository or solid models are used to provide data such as shape or scale of a spine or part thereof, or to provide a shape or an image for correlation.

In some embodiments, previous results are used as prior information (‘learning’).

Particular techniques, such as listed above and/or other image processing methods and tools are employed separately or in combination (collectively hereinafter ‘image processing’) to obtain information such as geometrical morphological or intrinsic characteristics of the spine or parts thereof and/or anatomical/geometrical relations therebetween as considered or determined suitable for a task. The particular techniques or tools and combinations thereof may be employed or adapted for particular tasks, and different techniques and combinations may vary for the same or similar tasks for better performance such as in speed, accuracy or reliability.

For clarity and brevity the use of image processing is implied in the following description without further citing or listing particular techniques, unless where deemed particularly worthwhile, without limiting and without excluding any other techniques or combination thereof.

In some embodiments, at least some of the image processing techniques and tools are activated and/or operate automatically. Optionally or alternatively the techniques and tools, or some or part thereof, are activated or operated manually, and are optionally modified or tuned under an operator's control.

### Overview

A general non-limiting overview of practicing the invention is presented below. The overview outlines practice of embodiments of invention and provides a basis relative to which variant and/or alternative and/or divergent embodiments are subsequently described.

### Modeling

A general non-limiting concept of typical embodiments of the invention is a quantitative modeling of the spine and/or parts thereof into an anatomically interrelated collection of properties comprising morphological and/or geometrical and/or intrinsic representation of the spine and/or parts thereof (‘model’).

In some embodiments, modeling comprises determining the spine shape and/or structure, for example, spinal curvature and torsion, vertebrae arrangement or spinal cord shape. In some embodiments, modeling comprises evaluating one or more geometrical elements or features or properties of the spine and/or components thereof, for example, dimensions of particular and/or representative parts of the vertebrae, geometrical properties of the intervertebral discs, cross-section area of the spinal canal or length of muscles or ligaments. In some embodiments, modeling comprises determining geometrical relationships between two or more components of the spine, for example, location of centers of vertebrae relative to one another or posteroanterior displacement or sagittal plane angle between neighboring vertebrae. In some embodiments, modeling comprises determining internal and/or intrinsic properties or characteristics of the spine and/or parts thereof, for example, water content of an intervertebral disc, density of muscles or ligaments or fat contents of a muscle.

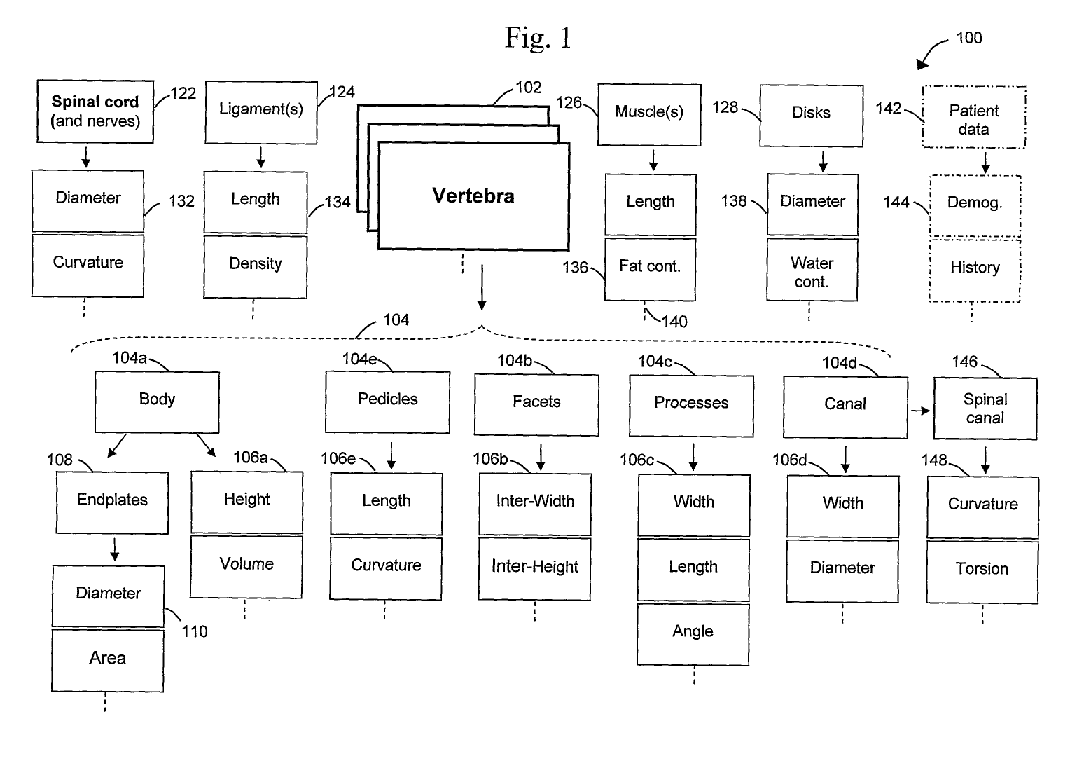

FIG. 1 schematically illustrates a simplified and abbreviated spine model 100 as a diagram of hierarchies and relations between elements of a spine, as a representation of a more comprehensive spine model, according to exemplary embodiments of the invention.

For brevity and clarity only few elements and properties are shown that represent further elements and properties deemed required for a spine model and assessment and/or diagnosis thereof. The dashed lines and brackets in FIG. 1, such as 104 and 140, indicate that more elements or properties are included, at least optionally, (e.g. more properties of muscles or more parts of vertebra).

The plurality of vertebrae 102 is at the hierarchy top level of model 100 (together with other elements, see below). The vertebrae elements 104 are at the next hierarchy level comprising (among other elements that are not shown) body 104a, facets 104b, processes 104c, pedicles 104e, and canal 104d. Elements 104 have respective properties 106 a level down the hierarchy of model 100. For example, body 104a has properties 106a (e.g. height, volume), facets 104b have properties 106b (e.g. inter-width and inter-height between facets), processes 104c have properties 106c (e.g. width, length and angle), pedicles 104e have properties 106e (e.g. length, curvature) and canal 104d (the hollow region of vertebrae 102) has properties 106d (e.g. width, diameter). In body 104a are other further parts 108 are defined or identified a level down the hierarchy of model 100, with properties 110 a level further down the hierarchy of model 100. For example, endplates 108 have properties 110 (e.g. diameter, area).

Also at the top level in the hierarchy of model 100 are ligaments 124 and muscles 126 which connect to vertebrae 102. Ligaments 124 and muscles 126 have properties 134 (e.g. length, density) and 136 (e.g. length, fat contents), respectively, a level down the hierarchy of model 100.

Also at the top level in the hierarchy of model 100 is spinal cord 122 which resides in the spinal canal constructed by canal parts 104d of vertebrae 102. Spinal cord 122 has properties 132 (e.g. diameter, curvature) a level further down the hierarchy of model 100.

Also at the top level in the hierarchy of model 100 are inter-vertebral discs 128. Discs 128 have properties 138 (e.g. diameter, water contents) level further down the hierarchy of model 100.

In some embodiments, the elements of canal 104d are constructed to form the spinal canal 146 with associated properties 148 (e.g. curvature and torsion).

The elements such as vertebrae 102, vertebra bodies 104a, facets 104b, processes 104c, pedicles 104e canals 104d and properties 106 thereof, and the other elements such as spinal cord 122, ligaments 124, muscles 126, discs 128, spinal canal 146 and properties thereof are anatomically and/or morphologically interrelated.

For simplicity and clarity, the relationships between elements are not indicated or partly indicated in FIG. 1. For example, vertebrae 102, spinal cord 122, ligaments 124, muscles 126 discs 128 and patient data 142 relationships are not indicated in FIG. 1.

In some embodiments, a plurality of models such as model 100 are constructed (e.g. a patient model and a reference model of healthy individuals). Typically, when two or more models, such as model 100, are used, for example, for comparison, the models are scaled and aligned with each other (normalized, see below).

Alongside the anatomical and morphological data (elements, properties), in some embodiments, patient data 142 is included in a model such as model 100. Patient data comprise information 142 such as demographic characteristics (e.g. age, gender, work and habits) and medical history (e.g. injuries, diseases) of the patient or current illness.

Optionally and particularly for a benchmark model, the model comprises supplementary values representing margins for normal distribution and optional standard deviations thereof (or other characteristic) in a given population, thereby assisting in comparing a patient conditions for abnormality, and allowing to evaluate a clinical significance of deviations from the model of patient's spine properties.

A model, such as model 100, is typically organized and/or implemented in a data structure or structures, typically in a memory device. For example, structured objects, linked-lists, linked or indexed arrays, relational data base, pre-set arrays (organized by the spine elements) or any technique or combination or techniques of data structure and organization in computer systems. In some embodiments, the model or parts thereof is formed in a ruled based model, expert system, machine learning model, statistical model, hybrid system and/or combinations thereof and/or other frameworks and combinations.

### Models Evaluation

In some embodiments of the invention, one or more anatomical parts and/or properties of a spine model of a person are evaluated to determine and/or distinguish anomalies such as irregularity, deformity or malfunction of the person spine.

In some embodiments of the invention, models of two or more spines, or parts thereof, are compared to determine and/or distinguish differences in respective anatomies and/or anatomical properties. Typically, in order to enable a comparison, the compared models, or parts thereof, are scaled to a common dominator and oriented to align with each other (normalized, see below).

In some embodiments, one or more of the reference models (e.g. benchmark model) are based on one or more spines or parts thereof which are considered or judged or classified as regular and/or ordinary and/or typical to represent a standard or normal spine (‘norm’). In some embodiments, a normal spine model is derived by combining (e.g. average, median, or other statistics) a plurality of spines and/or spine models, or components thereof, thereby reducing or eliminating personal variations, potentially achieving a model of benchmark spine.

In some embodiments, one or more of the reference or benchmark models are based on one or more spines which are derived from external sources such as anatomical repositories. For example, image banks (e.g. Essentials of Clinical Anatomy Image Bank, ISBN: 9780781743563, Lippincott Williams & Wilkins, 2002) or atlases (e.g. Gray's Anatomy: The Anatomical Basis of clinical Practice ISBN-13: 9780443066849 Elsevier Health Sciences, 2008), or solid three dimensional models (e.g. B Scientific GmbH, Rudorffweg 8, 21031 Hamburg, Germany). It should be emphasized that solid three-dimensional models as well as images or elements in an anatomical repository are not to be equated with the spine model as described and characterized above. Optionally, external sources comprise other prior knowledge such as geometrical information of sizes and shapes of a spine or parts thereof.

In some preferred embodiments, based on a model such as model 100, the determined conditions of a person's spine or of elements thereof and/or differences relative to another spine model (e.g. benchmark) are used for clinical assessment and/or diagnosis of a patient spine. For example, a condition is determined or decided or assesses as abnormal if the deviation or deviations (or standard deviation and/or other statistic and or derivative) are beyond a certain and/or preset and/or determined limit, or if a cumulation of deviations are beyond a limit while at least some individual deviations are below the limit. In some embodiments, clinical assessment and/or diagnosis of a patient spine comprises indicating a possible cause to and/or or likelihood of a back pain, where a likelihood of a back pain is, for example, estimating a time and/or physical activity that might eventually result in a back pain or increase in the risk of a back pain.

Optionally the assessment or the reference spine and/or model takes into consideration demographic peculiarities such as due to age and/or gender and/or race or clinical data such as the patient illness (e.g. inflammation or sub acute muscle weakness along with sub acute hyper-lordosis) or medical history (anamnesis).

In some embodiments, a prior model of a patient spine serves as a reference to track changes in the patient spine. For example, comparing models of a patient of different times provides indication or diagnosis or trending of the development of regression of the patient's spine disorder or other ailments.

It should be emphasized that comparing spines' anatomies, or models thereof, is or can be carried out by one or more techniques to obtain a measure of the difference or discrepancy or deviation between the anatomies or the models thereof. For example, form difference, form correlation (e.g. of curves) or geometrical difference (e.g. difference in diameters, lengths, curvature).

In some embodiments, the comparison is carried out according to the following formula that exemplifies muscles deviation:

$${{D}^{̑}}_{muscle} = \frac{{D}_{muscle} - {{Σ}_{{D}_{muslce}}}^{ } }{{σ}_{{D}_{muslce}}}$$

where D-caret is a normalized or unitless (dimensionless) measure of a deviation, D_{muscle} is a tested muscle, ΣD_{muscle} is the sum of a measure of the muscle of a reference model (e.g. muscles density, or distance to vertebra), and σ D_{muscle} is a standard deviation of D_{muscle}. It should be noted that for proper comparisons the compared properties values should be normalized such as described below.

### Operation Overview

Below are outlined operations for assessment of a spine of a patient.

(I) Obtaining image or images of one or more spines or parts thereof, using imaging equipment as described above.

(II) In some embodiments of the invention, preliminary procedures ('pre-processing') are used to aid in building the model and for identification of the spinal components. In some embodiments, the pre-processing comprises of image improvements (e.g. filtration or noise removal), and/or identification of body or trunk posture and spine identification and extraction. The identification is carried out by image processing, optionally aided by external sources.

(III) Identification of spine elements comprising (a) a hard tissues such as vertebrae and various osseous parts thereof, and (b) soft tissues such as intervertebral discs, spinal ligaments, spinal cord and optionally muscles and nerves. The identification is carried out by image processing, optionally aided by external sources.

(IV) Identification of elements, features, or properties of spine parts, such as disc nucleus or vertebra's body. The elements, features, or properties comprise also distinct points such as vertebra process' apex or muscle attachment site. The identification is carried out by image processing, optionally aided by external sources.

(V) Segmentation of elements and features of the spine to regions such as bone, spinal canal, intervertebral discs, spinal ligaments, spinal cord or muscles. The identification is carried out by image processing, optionally aided by external sources. The segmentation is carried out by image processing, optionally aided by external sources.

(VI) Calculation of geometrical morphological characteristics (properties) of elements and features as derived from the segmentation, such as height, width, cross sectional area, orientation. The calculation is carried out by image processing, optionally aided by external sources.

(VII) Calculation of elements characteristics as derived from the segmentation, such as density of an element or part thereof or texture of an element. For each element and feature calculate from the segmented volume. The calculation is carried out by image processing, optionally aided by external sources.

In some embodiments of the invention, the operations, at least (III to VII) are carried out automatically. Optionally, at least some of the operation are manually initiated or controlled.

In some embodiments of the invention, an operator is provided with tools and/or or mechanisms for editing the identifications of elements (e.g. specific intervertebral disc, vertebra, or muscle), editing identification of a feature or part of an element (e.g. disc nucleus, vertebra's body, vertebra process' apex, or muscle attachment site), or editing or modifying the identification of an element or a segmentation thereof.

(VIII) Constructing a model of a spine based on the identified and calculated (evaluated) element. Optionally a benchmark or reference model is constructed using data acquired from one or more individuals to evaluate or determine differences as described above.

(IX) Assessment and/or diagnosis of a patient's spine based on the model of the patient's spine, optionally and additionally based also on other resources such as a reference model and/or other clinical or demographic data of the patient.

(X) Presentation to an operator or clinician the patient's spine, such as the patient's spine model, optionally with other data such as a reference model or clinical data for assessment and/or diagnosis of the patient spine.

In some embodiments, operations (IX) and (X) are combined, at least partially. Optionally, operation (IX) is reduced to operation (X).

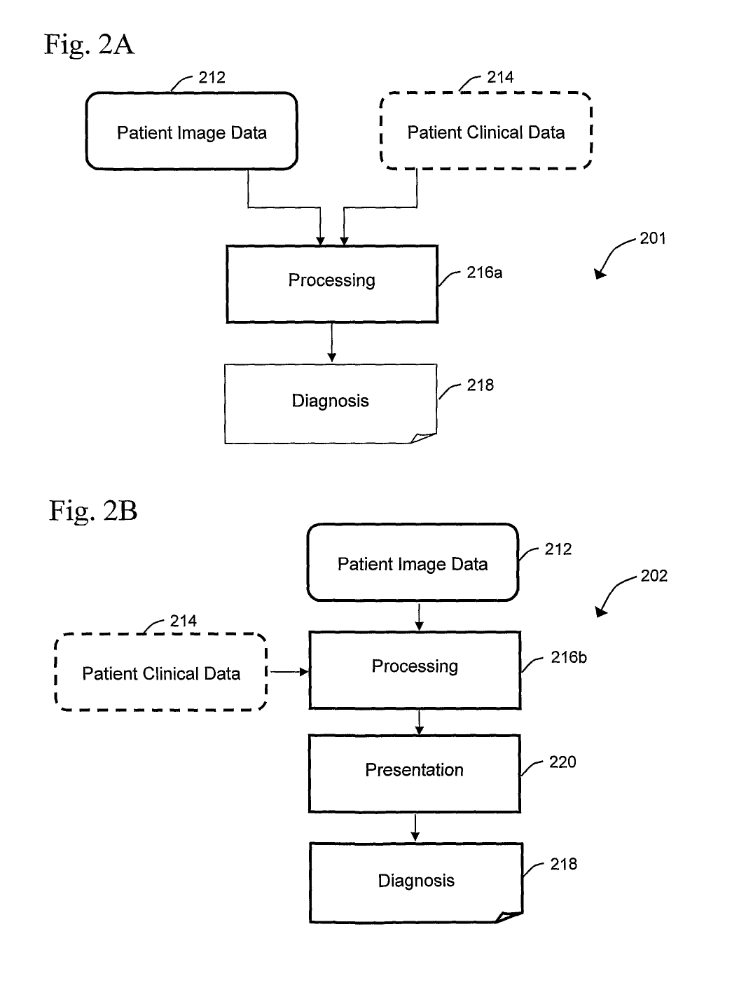

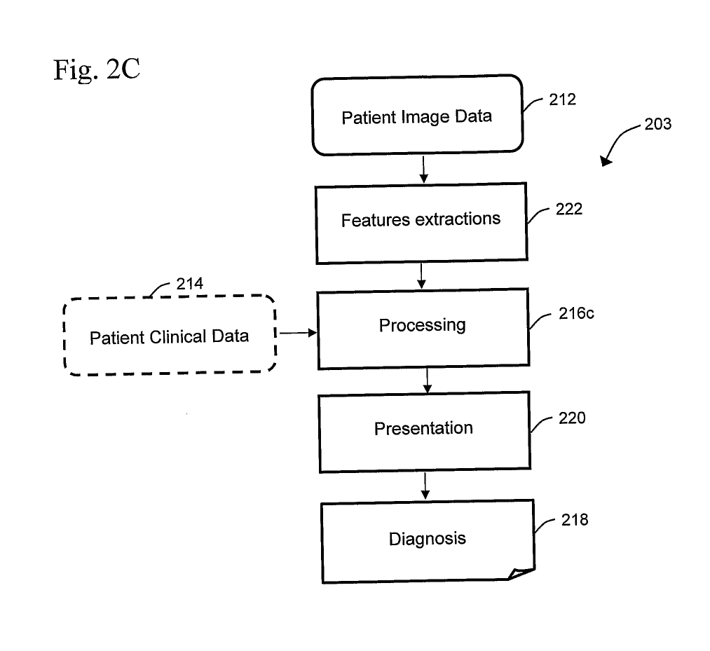

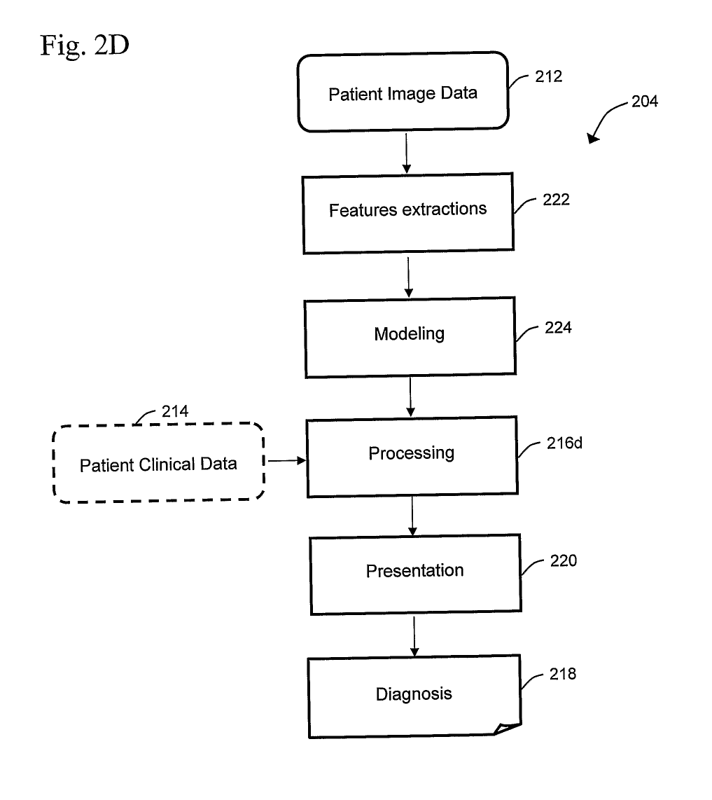

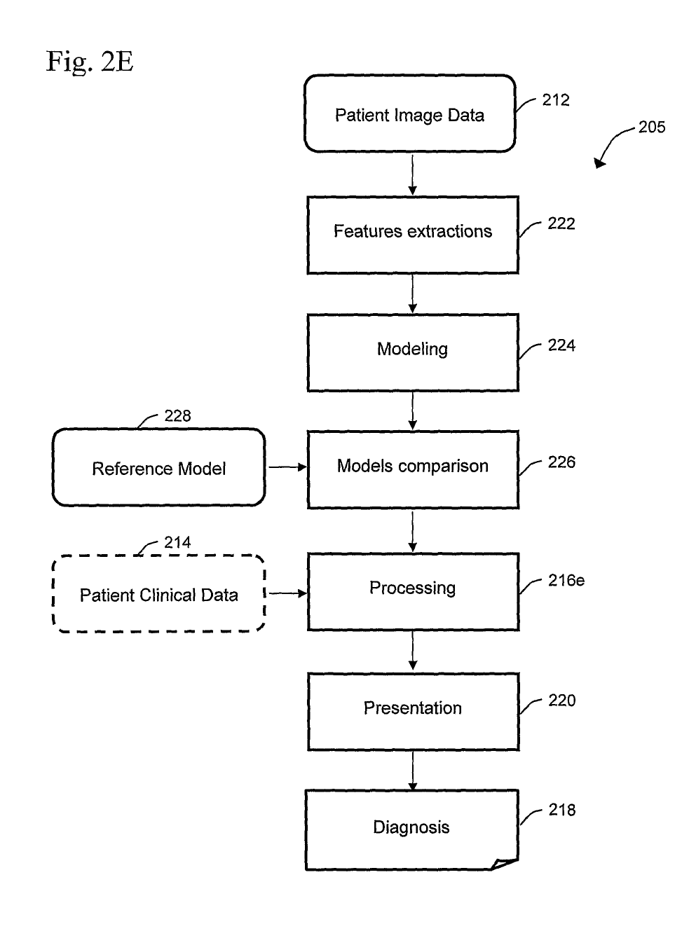

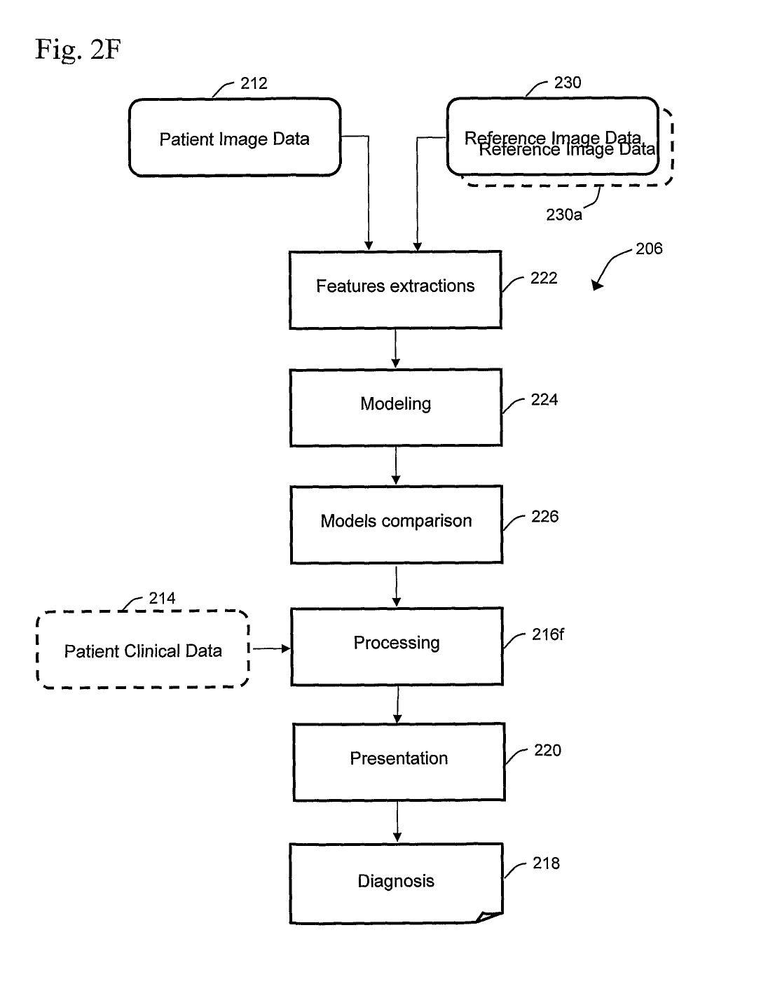

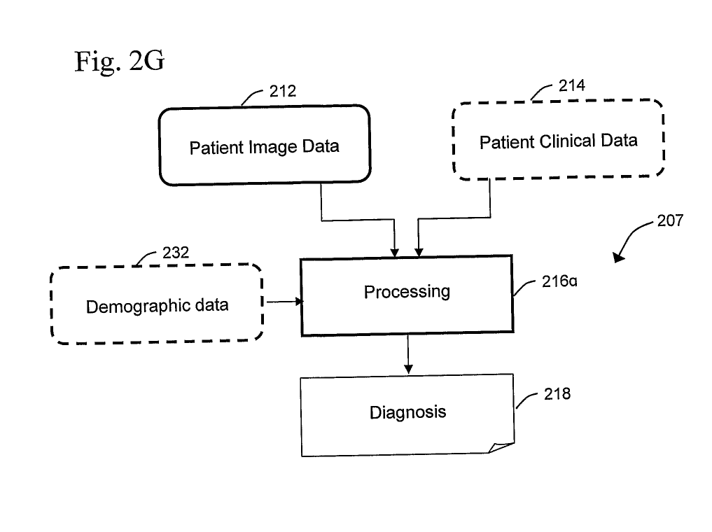

FIGS. 2A-G illustrates various non-limiting exemplary overviews of data and operations for assessments of a patient's spine.

FIG. 2A illustrates a chart 201 schematically outlining data and actions for assessment of a patient's spine, according to exemplary embodiments of the invention.

The patient spine is acquired (imaged) and the image or images are provided (212). Optionally, some clinical data of the patient are also provided (214). The provided data is processed (216a), analyzing the spine anatomy and morphology. Optionally, the analysis comprises comparison to other spines or models thereof. Based on the analysis results, and optionally with respect to the provided clinical data, a diagnosis (or clinical assessment) of the patient's spine is provided (218). In some embodiments, assessment (218) is based on and/or aided by presentation of the patient's spine of part thereof, optionally with a reference spine or other data (e.g. patient's illness), and optionally the presented data or other data of the patient is edited.

FIG. 2B illustrates a chart 202 schematically outlining a further action for assessment of a patient's spine with respect to chart 201, with modified or simplified processing (216b relative to 216a), according to exemplary embodiments of the invention.

Chart 202 illustrates the further operation of presentation or display (220) of a patient's spine or part thereof, optionally together with some additional data such clinical information. In some embodiments, a patient spine or part thereof is presented together with a reference spine or part thereof, demonstrating differences or anomaly of the patient's spine with respect to the reference. The presentation is manually and/or automatically initiated and is interactively controlled by an operator for convenient viewing and examination for providing an assessment or diagnosis of the patient' spine (e.g. 218).

In preferred embodiments, the presented data or part thereof is modified graphically or alphanumerically (e.g. in tables or forms), typically interactively such as by a keyboard or mouse or any suitable device or method. For example, smoothing a rugged surface due to image processing, tuning the alignment of a spine to a reference spine or fixing a numerical value such as a property of a spine element or margins of a norm.

In some embodiments, measurements and calculations or other derivations are performed on or based on the presented data, optionally based also on non-presented data. For example, measurement of areas, volumes or densities or misalignments with a reference. In some embodiments, data related to the patient or spine thereof is added or removed. For example, adding an element or property based on another modality (e.g. US after feature extraction), adding or removing annotations on a model or part thereof, adding measurements results, providing an assessment or diagnosis, modifying a previous diagnosis or removing an indication of an illness.

Modified processing (216b) employs the extracted elements for the presentation (220) and diagnosis (218), optionally employing the patient clinical data (214).

FIG. 2C illustrates a chart 203 schematically outlining a further action for assessment of a patient's spine with respect to chart 202, with modified or simplified processing (216c relative to 216b), according to exemplary embodiments of the invention.

Chart 202 illustrates the further operation of features extraction to obtain elements and properties of a spine of parts thereof (222). Modified processing (216c) employs the extracted elements for the presentation and diagnosis, optionally employing the patient clinical data (214).

FIG. 2D illustrates a chart 204 schematically outlining a further action for assessment of a patient's spine with respect to the chart 203, with modified or simplified processing (216d relative to 216c), according to exemplary embodiments of the invention.

Chart 204 illustrates another operation of modeling the patient spine by the extracted elements (224) and providing a model of the patient spine. Modified processing (216d) employs the model for presentation and diagnosis, optionally employing the patient data (214).

FIG. 2E illustrates a chart 205 schematically outlining other data and action for assessment of a patient's spine with respect to chart of 204, with modified processing (216e relative to 216d), according to exemplary embodiments of the invention.

As illustrated in chart 205, a reference model is provided (228) and compared with the patient's model (226). The comparison results are processes (216e) and optionally presented (220). A diagnosis (or clinical assessment) of the patient's spine is provided (218), optionally with respect to the provided clinical data (214).

FIG. 2F illustrates a chart 206 schematically outlining a further action for assessment of a patient's spine with respect to chart of 201, with modified processing (216f relative to 216e), according to exemplary embodiments of the invention.

Instead of providing a reference model as in chart 205, in chart 206 an image data of a reference spine (or other data of the reference, such as measurements) are provided (230). The anatomical and morphological features of both the patient data (212) and the reference data (230) are extracted (212), and both respective models are constructed (224) and compared (226). In some embodiments, data of a plurality of spines are provided (e.g. 230a) and a reference spine model is constructed based on the data of the plurality of spines (or models thereof, at least partially).

FIG. 2G illustrates a chart 207 schematically outlining a further action for assessment of a patient's spine with respect to chart of 201, with modified processing (216g relative to 216a), according to exemplary embodiments of the invention.

Since persons of different demographic origins or characteristic may exhibit variations in the spine anatomy or morphology that may be considered as anomalous in other populations, demographic data is provided (232) for processing.

In some preferred embodiments the diagnosis or clinical assessment (e.g., 218) and/or image data and/or other input or generated data are provided for review and possible editing (e.g. 220).

It should be emphasized that the variations described in FIGS. 2A-G are non-limiting and may vary further and combined in any manner for providing assessment or diagnosis of the patient's spine condition.

### Measurements and Normalization

Below are described, among others, geometrical measurements of elements or features of a spine and parts thereof. In order to facilitate sensible comparisons between two or more spines (or models thereof), or quantitative assessment of a spine or elements thereof (or spine model), respective spines or elements, or measures thereof, are normalized (or registered) as follows.

An ingredient of normalization is establishing a common scale and geometrical measurements are normalized thereto, for example, a multiplication by a factor to share a common denominator.

According to particular cases, linear (uniform) or non-linear (non-uniform) scaling operations are performed, optionally only on a part or parts of the spine or elements thereof.

In preferred embodiments, the normalization provides a coordinate system independent of the patient or of any other source.

In some embodiments, prior or subsequent to scaling, orientation matching is performed as described below.

Some non-limiting candidate bases for normalization are described below.

### Distance Basis

(i) Vertebra body anterior posterior distance for specific vertebra (e.g. T12, L3, L5) or as mean value over several vertebrae (e.g. T12 to L5).

(ii) Vertebra body lateral distance for specific vertebra (e.g. T12, L3, L5) or as mean value over several vertebrae (e.g. T12 to L5).

(iii) Vertebra body endplate effective diameter (diameter calculated using endplate cross sectional area, and assuming the endplate is a circle) either for specific vertebra and endplate (e.g. bottom endplate of T12, top endplate of L3, bottom endplate of L5) or as mean value over several vertebrae (e.g. top and bottom endplates of T12 to L5).

(iv) Distance along the spinal canal centerline (e.g. distance between top of L1 to bottom of L5 along the spinal canal centerline).

(v) Patient height.

(vi) Different scale in axial plane (XY direction) of the patient coordinates, for example L3 vertebra body anterior-posterior diameter to, in scale different from (i)-(v) above.

(vii) L3 vertebra body effective diameter (e.g. the mean bounding region dimension such as one-half of the sum of the short and long axis lengths) to scale in axial plane (XY direction), for example, in scale different from (i)-(v) above.

### Area Basis

(i) Vertebra body endplate (discal surface) area—either for specific vertebra and endplate (e.g. bottom endplate of T12, top endplate of L3, bottom endplate of L5) or as mean value over several vertebrae (e.g. top and bottom endplates of T12 to L5).

(ii) Spinal canal average or segmental average cross section area—the cross section area of the spinal canal either at a specific location, average along the entire canal, or average along a segment (e.g. average of cross section from L1 to L5).

### Volume Basis

(i) The volume of a specific vertebra body.

(ii) The average volume of several vertebra bodies (e.g. all vertebra bodies C3-L5, or the lumbar vertebra bodies L1-L5).

### Alignment

In some embodiments, normalization comprises also matching of orientation (also referred to as registration). For example, the spine curve is rotated such the anterior-posterior line is oriented on common axis (e.g. the Y axis) by projecting the curve on the XY plane and finding the major axis of points' distribution (e.g. using Principal Components Analysis) and the orientation (angle) of major axis, followed by rotation of the curve such that the major axis will align with the Y (anterior-posterior) direction.

### Elements of a Vertebra

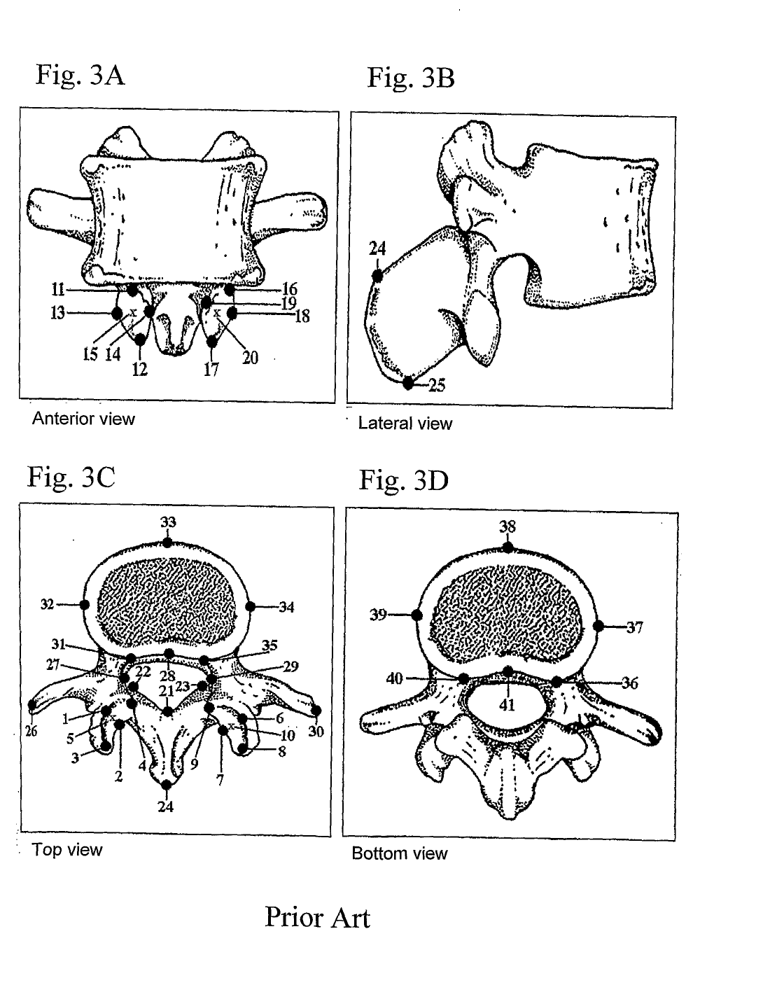

FIGS. 3A-D schematically illustrate a vertebra from four view sides (views), anterior, lateral, top and bottom, respectively. Some particular elements or features are indicated with rounded circles and labeled with numerals 1-41.

Table-1 below briefly describes each labeled feature, where ‘AL-n’ stand for a numeral label, e.g. AL-1 stands for a feature labeled as 1.

TABLE 1

| | |
|---|---|
| AL-1 | Superior border of left superior articular facet |
| AL-2 | Inferior border of left superior articular facet |
| AL-3 | Lateral border of left superior articular facet |
| AL-4 | Medial border of left superior articular facet |
| AL-5 | Center of left superior articular facet |
| AL-6 | Superior border of right superior articular facet |
| AL-7 | Inferior border of right superior articular facet |
| AL-8 | Lateral border of right superior articular facet |
| AL-9 | Medial border of right superior articular facet |
| AL-10 | Center of right superior articular facet |
| AL-11 | Superior border of right inferior articular facet |
| AL-12 | Inferior border of right inferior articular facet |
| AL-13 | Lateral border of right inferior articuLar facet |
| AL-14 | Medial border of right inferior articular facet |
| AL-15 | Center of right inferior articular facet |
| AL-16 | Superior border of left inferior articular facet |
| AL-17 | Inferior border of left inferior articular flhcet |
| AL-18 | Lateral border of left inferior articular facet - |
| AL-19 | Medial border of left inferior articular facet |
| AL-20 | Center of left inferior articular facet |
| AL-21 | Anterosuperior border of spinous process - |
| AL-22 | External border of superior left lanina |
| AL-23 | External border of superior right lamina - - |
| AL-24 | Posterosuperior border of spinous process --- |
| AL-25 | Posteroinferior border of spinous process |
| AL-26 | External border of left transverse process |
| AL-27 | Left superior border of vertebral canal |
| AL-28 | Anterosuperior border of vertebral canal |
| AL-29 | Right superior border of vertebral canal |
| AL-30 | External border of right transverse process |
| AL-31 | Left posterior border of superior vertebral body |
| AL-32 | Left border of superior vertebral body |
| AL-33 | Anterior median border of superior vertebral body |
| AL-34 | Right border of superior vertebral body |
| AL-35 | Right Posterior border of superior vertebral body |
| AL-36 | Left posterior border of inferior vertebral body |
| AL-37 | Left_border_of inferior_vertebral_body |
| AL-38 | Anterior median border of inferior vertebral body |
| AL-39 | Right border of inferior vertebral body |
| AL-40 | Right Posterior border of inferior vertebral body |
| AL-41 | Posterior median border of inl vertebral body |

### Vertebrae Measurements Examples

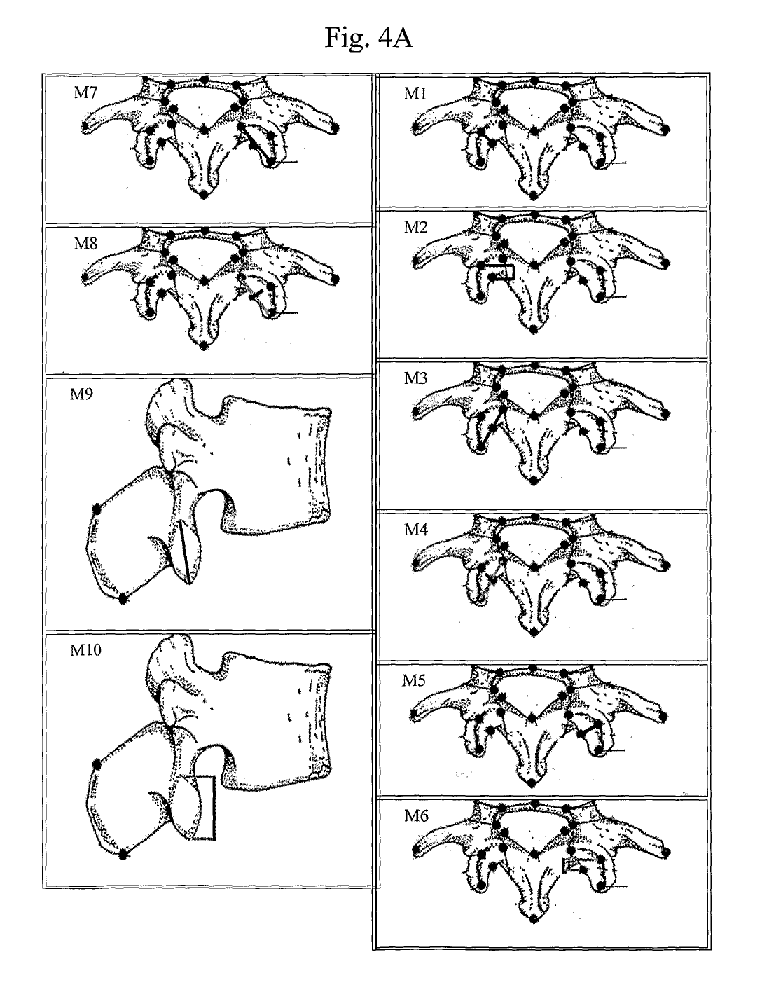

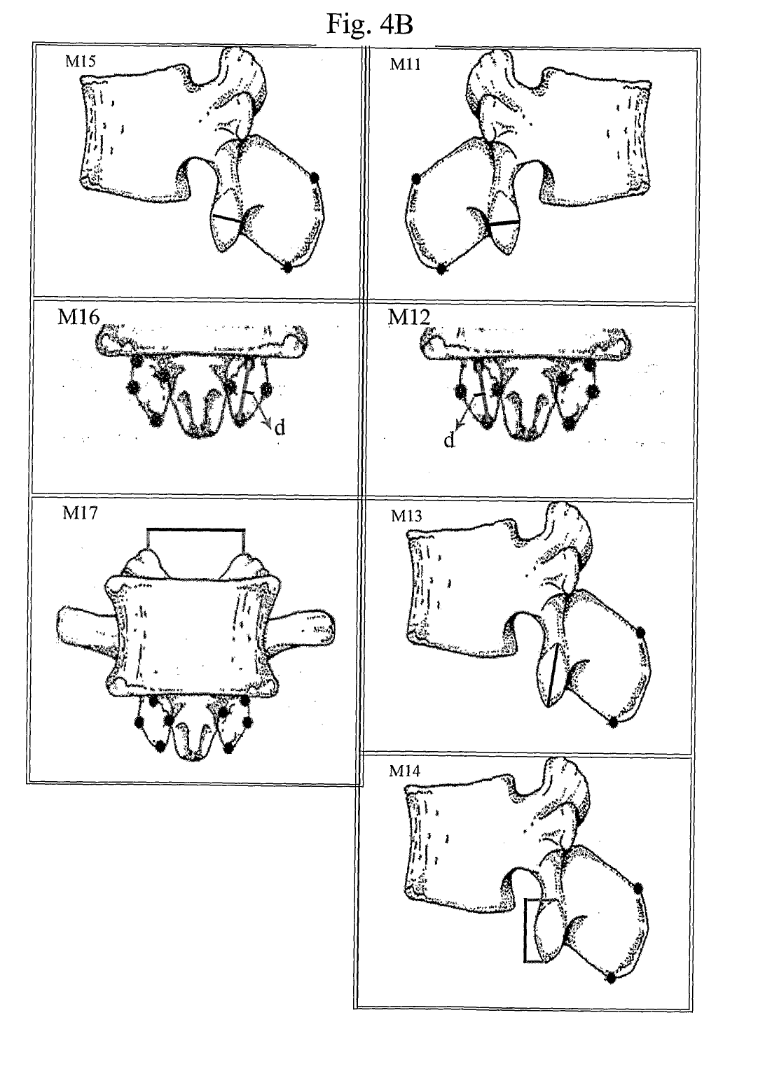

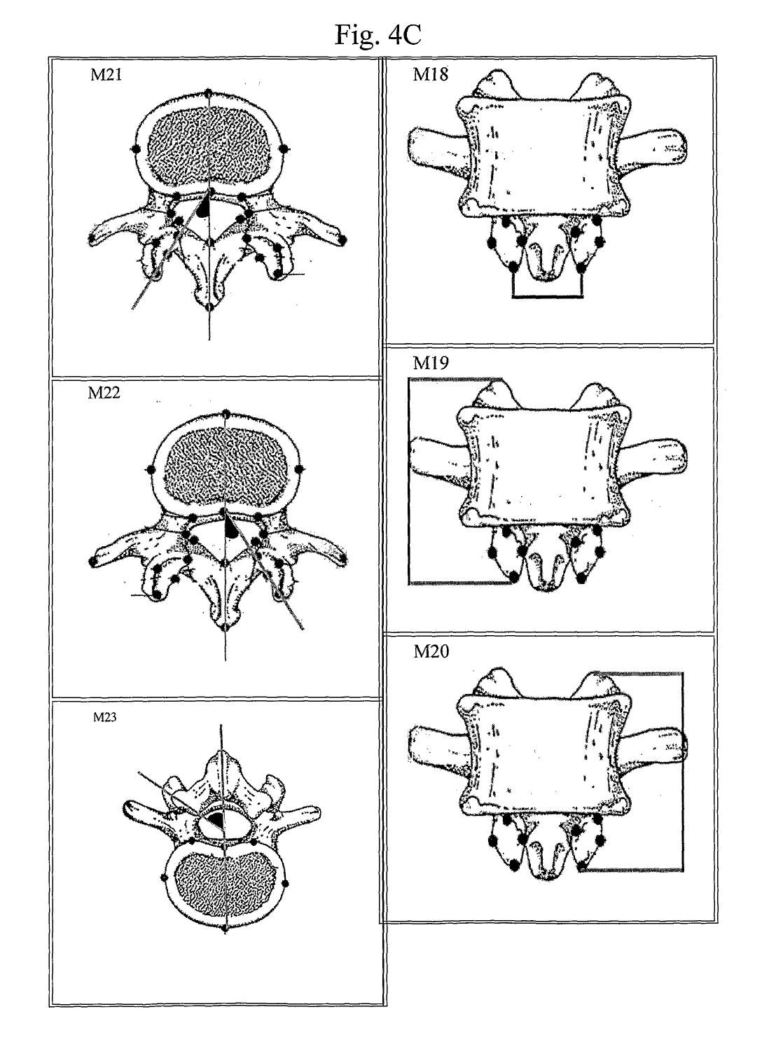

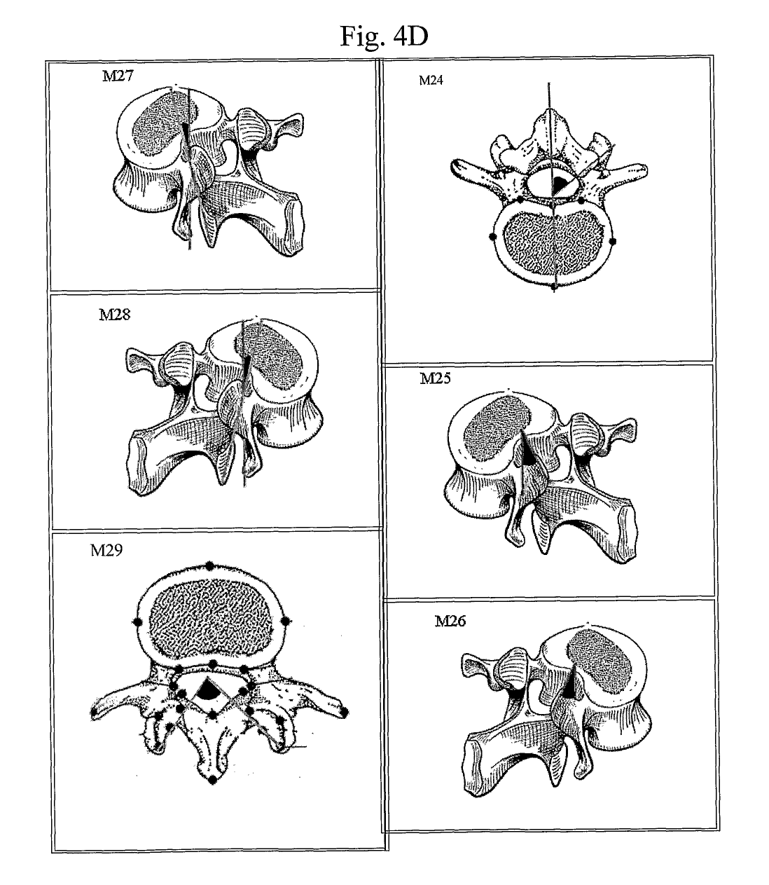

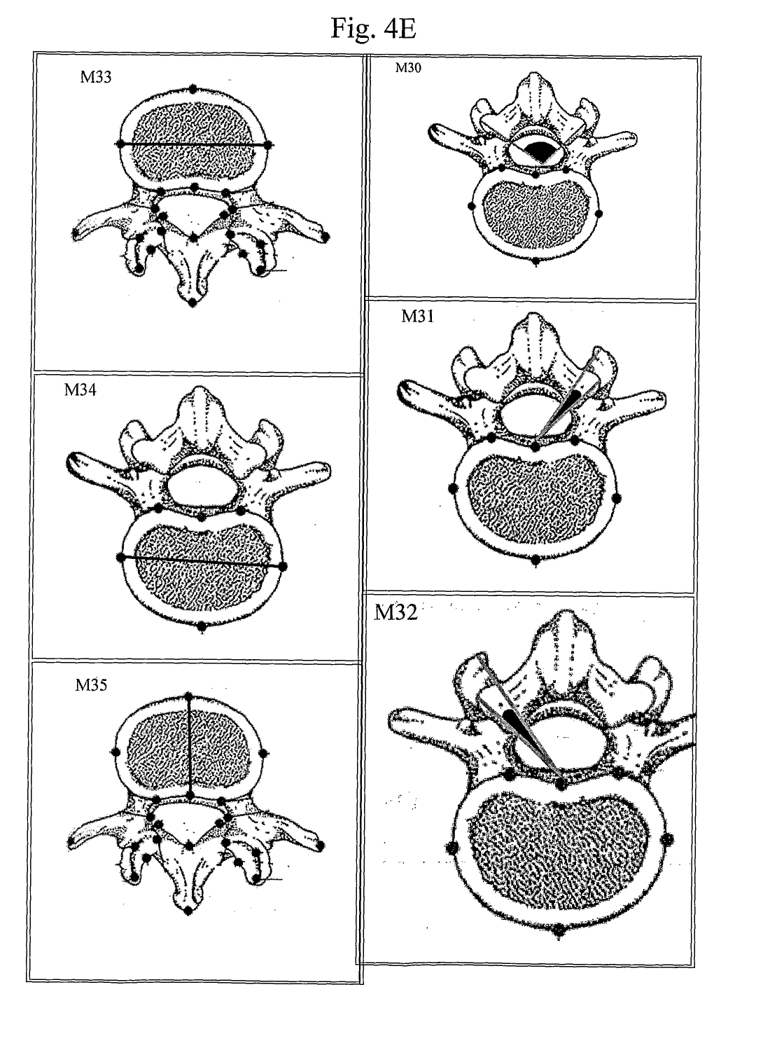

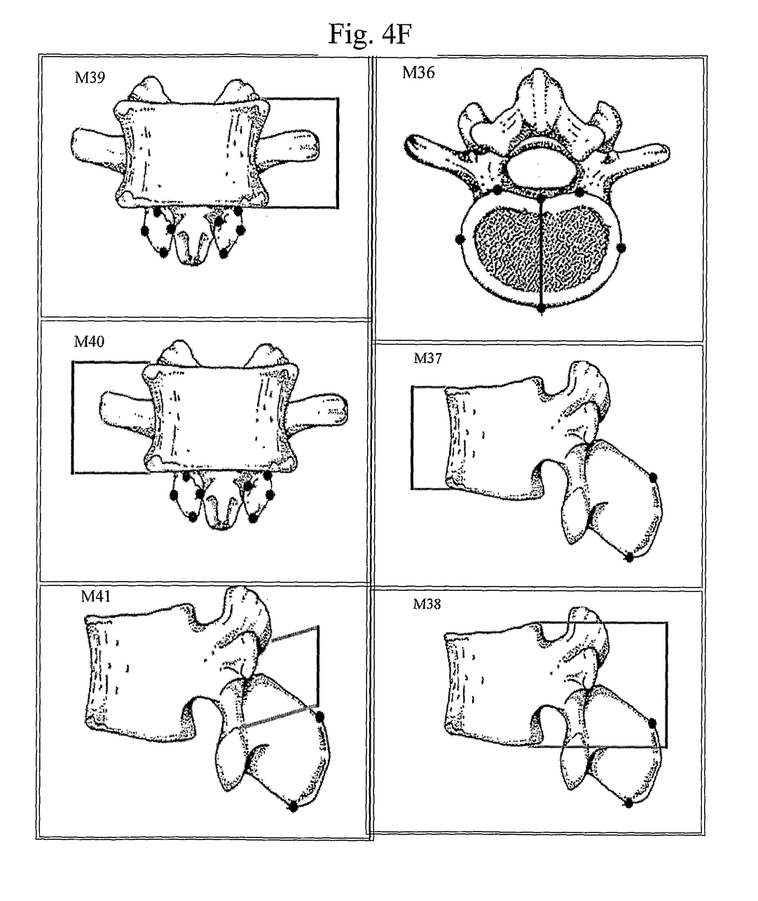

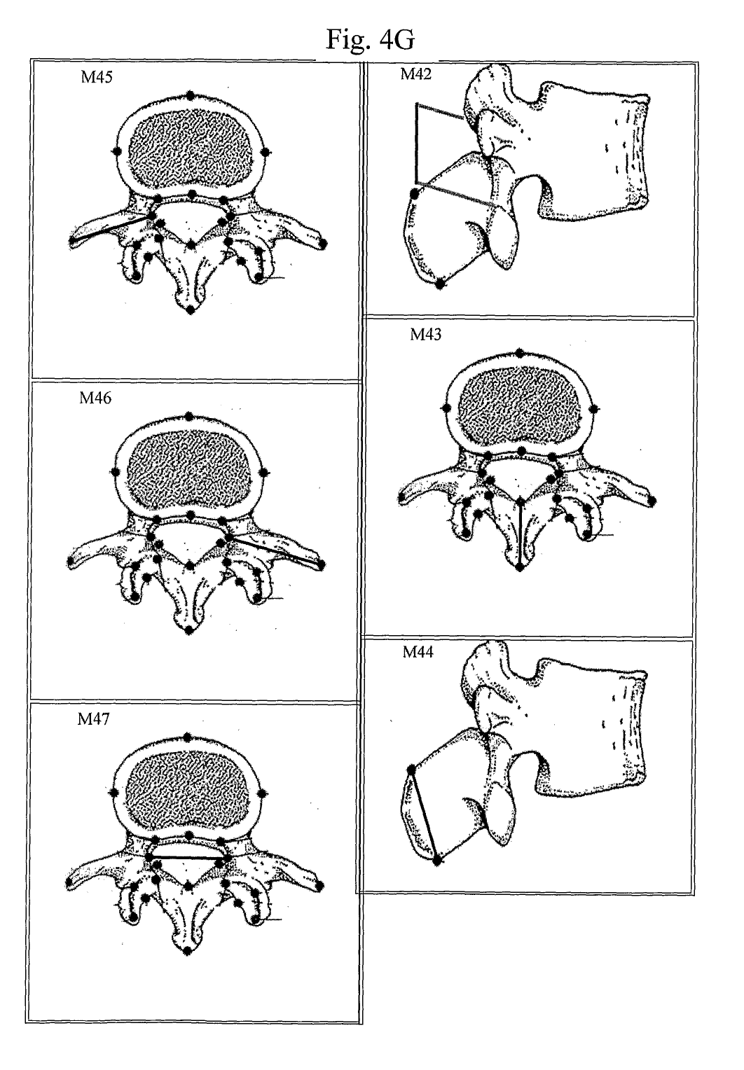

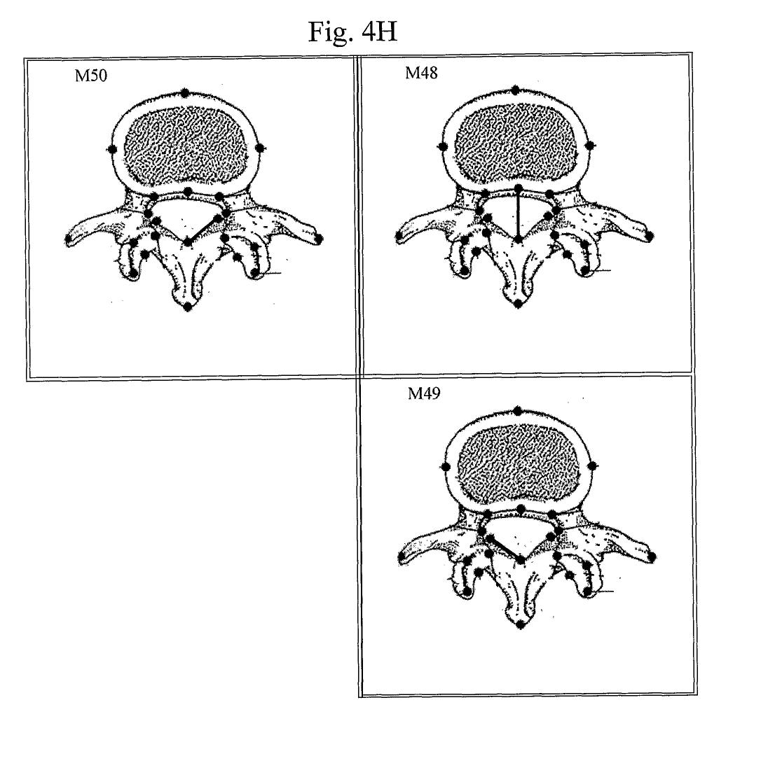

Table-2 and corresponding FIGS. 4A-H schematically illustrate and briefly describe various measurements (metrics) of properties or elements of vertebrae, where identification tags (e.g. M1, M2) indicate the correspondence between Table-2 and FIGS. 4A-H.

TABLE 2

| | | Measurement definition in relation to anatomical |
|---|---|---|
| Mark | Measurement Name | landmarks (AL) of Table-1 and FIGS. 3A-D. |
| M1 | Left superior facet | Distance between superior and inferior borders of left |
| | length (LSFL) | superior articular facet (AL-i and AL-2) |
| M2 | Left superior facet | Projected distance (in the frontal plane) |
| | height (LSFH) | between superior and inferior borders of left |
| | | superior articular facet (AL-i and AL-2) |
| M3 | Left superior facet width | Distance between lateral and medial borders of left |
| | (LSFW) | superior articular facet (AL-3 and AL-4) |
| M4 | Left superior facet depth | Distance between the center of left superior articular facet |
| | (LSFD) | (AL-5) to the line of LSFW (M3) |
| M5 | Right superior facet | Distance between superior and inferior borders of right |
| | length (RSFL) | superior articular facet (AL-6 and AL 7) |
| M6 | Right superior facet | Projected distance (in the frontal plane) |
| | height (RSFH) | between superior and inferior borders of right |
| | | superior articular facet (AL-6 and AL-7) |
| M7 | Right superior facet | Distance between lateral and medial borders of right |
| | width (RSFW) | superior articular facet (AL-8 and AL-9) |
| M8 | Right superior facet | Distance between the center of right superior articular facet |
| | depth (RSFD) | (AL-10) to the line of RSFW (M7) |
| M9 | Right inferior facet | Distance between superior and inferior borders of right |
| | length (RIFL) | inferior articular facet (AL-li and AL 12) |
| M10 | Right inferior facet | Projected distance (in the frontal plane) |
| | height (RTFH) | between superior and inferior borders of right |
| | | inferior articular facet (AL-i 1 and AL-12) |
| M11 | Right inferior facet | Distance between lateral and medial borders of |
| | width (RIFW) | right inferior articular facet (AL-13 and AL-14) |
| M12 | Right inferior facet | Distance (d) between the center of right inferior articular |
| | depth (RIFD) | facet (AL-15) and the line of RIFW (Mu) |
| M13 | Left inferior facet length | Distance between superior and inferior borders of left |
| | (LIFL) | inferior articular facet (AL- 16 and AL 17) |
| M14 | Left inferior facet height | Projected distance (in the frontal plane) |
| | (LIFH) | between superior and inferior borders of left |
| | | inferior articular facet (AL-16 and AL-i7) |
| M15 | Left inferior facet width | Distance between lateral and medial borders of left inferior |
| | (LIFW) | articular facet (AL- 18 and AL- 19) |
| M16 | Left inferior facet depth | Distance (d) between the center of left inferior articular |
| | (LIFD) | facet (AL-20) and the line of LIFW (M15) |
| M17 | Superior interfacet width | Projected distance (in the sagittal plane) |
| | (SFFW) | between the superior borders of left and right |
| | | superior articular facets (AL-i and AL-6) |
| M18 | Inferior interfacet with | Projected distance (in the sagittal plane) |
| | (IFFW) | between the inferior borders of left and right |
| | | inferior articular facets (AL-12 and AL-17) |
| M19 | Left interfacet height | Projected distance (in the frontal plane) between superior |
| | (LFFH) | border of left superior articular facet (AL-i) and inferior |
| | | border of left inferior articular facet (AL-17) |
| M20 | Right interfacet height | Projected distance (in the frontal plane) between superior |
| | (RFFH) | border of right superior articular facet (AL-6) and inferior |
| | | border of right inferior articular facet (AL- 12) |
| M21 | Left superior | The angle formed (in the sagittal plane) at the intersection |
| | transverse facet | between the lines defining left superior facet width (M3) |
| | angle (LSTFA) | and superior vertebral body length (M35) |
| M22 | M22 Right superior | The angle formed (in the sagittal plane) at the intersection |
| | transverse facet | between the lines defining right superior facet width (M7) |
| | angle (RSTFA) | and superior vertebral body length (M35) |
| M23 | Left inferior | The angle formed (in the sagittal plane) at the intersection |
| | transverse facet | between the lines defining left inferior articular facet |
| | angle (LITFA) | (LIAF) width (M15) and inferior vertebral body length |
| | | (M36) |
| M24 | Right inferior transverse | The angle formed (in the sagittal plane) at the intersection |
| | facet angle (RITFA) | between the lines defining right inferior articular facet |
| | | (RIAF) width (Ml 1) and inferior vertebral body length |
| | | (M3 6) |
| M25 | Left superior | The superior angle formed (in the frontal plane) at the |
| | longitudinal facet angle | intersection between the lines defining left superior |
| | (LSLFA) | articular facet length (Ml) and posterior vertebral body |
| | | height (M38) |
| M26 | Right inferior | The superior angle formed (in the frontal plane) at the |
| | longitudinal facet angle | intersection between the lines defining right inferior |
| | (RILFA) | articular facet length (M9) and posterior vertebral (M3 8) |
| M27 | Right superior | The superior angle formed(in the frontal plane) at the |
| | longitudinal facet angle | intersection between the lines defining right superior |
| | (RSLFA) | articular facet length (M5) and posterior vertebral body |
| | | height (M3 8) |
| M28 | Left inferior longitudinal | The superior angle formed(in the frontal plane) at the |
| | facet angle (LILFA) | intersection between the lines defining left inferior articular |
| | | facet length (M13) and posterior vertebral body height |
| | | (M38) |
| M29 | Transverse superior | Angle formed (in the sagittal plane) at the intersection |
| | interfacet angle (TSFFA) | between the lines defining left superior articular facet |
| | | width (M3) and right superior articular facet width (M7) |
| M30 | Transverse inferior | Angle formed (in the sagittal plane) at the intersection |
| | interfacet angle (TIFFA) | between the lines defining left inferior articular facet |
| | | (LIAF) width (M15) and right inferior articular facet |
| | | (RIAF) width (M11) |
| M31 | Left transverse torsion | Angle formed (in the sagittal plane) at the intersection |
| | interfacet angle | between line A defining the left superior articular facet |
| | (LTTFFA) | width (M3) and line B defining the left inferior articular |
| | | facet width (M15) |
| M32 | Right transverse torsion | Angle formed (in the sagittal plane) at the intersection |
| | interfacet angle | between line A defining the right superior articular facet |
| | (RTTFFA) | width (M7) and line B defining the right inferior articular |
| | | facet width (M11) |
| M33 | Superior vertebral body | Projected distance (in the frontal plane) between left and |
| | width | right borders of superior vertebral body (AL-32 and AL- |
| | (SVBW) | 34) |
| M34 | Inferior vertebral body | Projected distance (in the frontal plane) between left and |
| | width | right borders of inferior vertebral body (AL-37 and AL-39) |
| | (IVBW) | |
| M35 | Superior vertebral body | Projected distance (in the sagittal plane) between anterior |
| | length (SVBL) | and posterior borders of superior vertebral body (AL-33 |
| | | and AL-28) |
| M36 | Inferior vertebral body | Projected distance (in the sagittal plane) between anterior |
| | length | and posterior borders of inferior vertebral body (AL-3 8 |
| | (IVBL) | and AL-4 1) |
| M37 | Anterior vertebral body | Projected distance (in the frontal plane) between anterior |
| | height | central borders of superior and inferior vertebral body (AL- |
| | (AVBH) | 33 and AL-38) |
| M38 | Posterior vertebral body | Projected distance (in the frontal plane) between posterior |
| | height | central borders of superior and inferior vertebral body (AL- |
| | (PVBH) | 28 and AL- 41) |
| M39 | Left vertebral body | Projected distance (in the frontal plane) between left |
| | height (LVBH) | borders of superior and inferior vertebral body (Al-32 and |
| | | AL-3 7) |
| M40 | Right vertebral body | Projected distance (in the frontal plane) between right |
| | height (RVBH) | borders of superior and inferior vertebral body (AL-34 and |
| | | AL-39) |
| M41 | Left isthmus length | Projected distance (in the frontal plane) between inferior |
| | (LIL) | border of left superior articular facet (AL-2) and superior |
| | | border of left inferior articular facet (AL- 16) |
| M42 | Right isthmus length | Projected distance (in the frontal plane) between inferior |
| | (RIL) | border of right superior articular facet (AL-7) and superior |
| | | border of right inferior articular facet (AL- 1 1) |
| M43 | Spinous process length | Projected distance (in the frontal plane) |
| | (SPL) | between anterior and posterior superior borders |
| | | of spinous process (AL-2 1 and AL-24) |
| M44 | Spinous process height | Projected distance (in the sagittal plane) between posterior |
| | (SPH) | superior and inferior borders of spinous process (Al-24 and |
| | | Al-25) |
| M45 | Left transverse process | Projected distance (in the sagittal plane) between external |
| | length (LTPL) | border of left transverse process (AL-26) and left superior |
| | | border of vertebral canal (AL-27) |
| M46 | Right transverse process | Projected distance (in the sagittal plane) between external |
| | length (RTPL) | border of right transverse process (AL-29) and right |
| | | superior border of vertebral canal (AL-30) |
| M47 | Vertebral canal superior | Distance between left superior border of vertebral canal |
| | width (VCSW) | (AL-27) and right superior border of vertebral canal AL-30 |
| M48 | Vertebral canal superior | Projected distance (in the frontal plane) between anterior |
| | length (VCSL) | superior border of spinous process (AL- 21) and anterior |
| | | superior border of vertebral canal (AL-28) |
| M49 | Left superior laminar | Projected distance (in the frontal plane) between anterior |
| | length | superior border of spinous process (AL-21) and inferior |
| | (LSLL) | border of left superior facet (AL-2) |
| M50 | Right superior laminar | Projected distance (in the frontal plane) between anterior |
| | length | superior border of spinous process (AL-2 1) and inferior |
| | (RSLL) | border of right superior facet and AL-7 |

### Image Processing and Segmentations Examples

Some non-limiting examples of features extraction is provided below demonstrating how the anatomy and/or morphology of the spine can be analyzed and measured as described further below.

### Vertebra

The examples below for vertebrae extraction are based on T. Klinder, J. Ostermann, M Ehm, A. Franz, R. Kneser, C. Lorenz, “Automated model-based vertebra detection, identification, and segmentation in CT images, “Medical Image Analysis, In Press., and J. Yao, S. O'Connor, and R. Summers, “Automated spinal column extraction and partitioning,” Biomedical Imaging: Nano to Macro, 2006. 3rd IEEE International Symposium on, 2006, pp. 390-393.

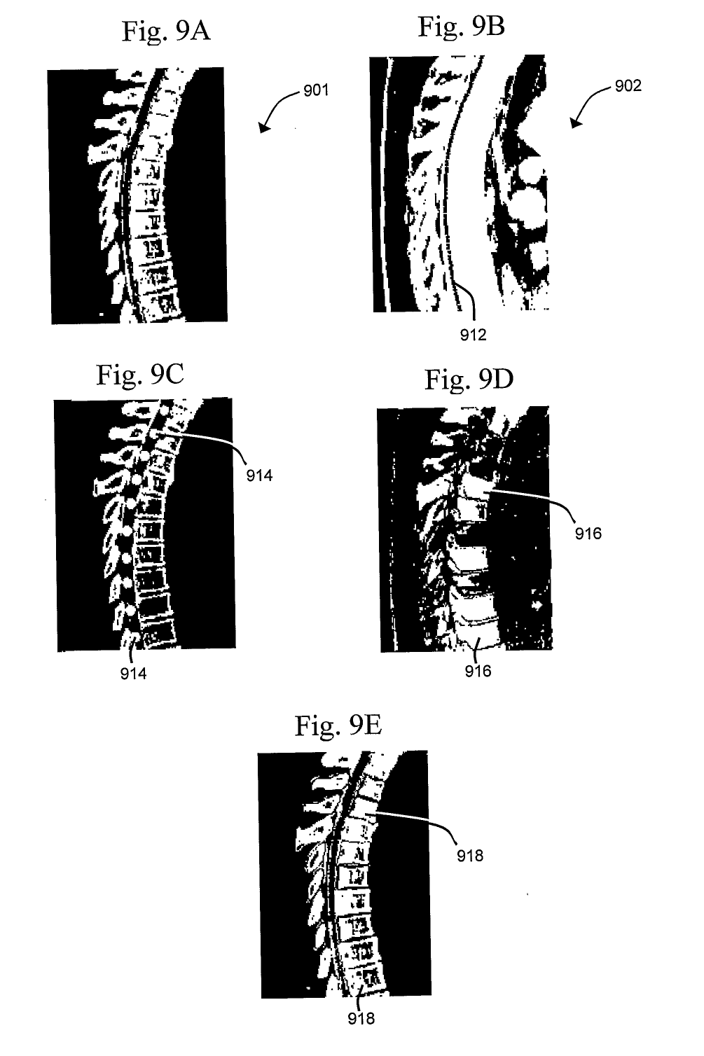

FIG. 9A illustrates a two-dimensional view 901 of a volumetric CT study of a spine after some pre-processing such as thresholding and region growing and conversion to black and white. For example, a threshold of 200 HU is used to mask out the bone pixels and then a connected component analysis is conducted on the bone mask and the largest connected blob in the center of the image is retained as the initial point for spinal canal segmentation. The resultant image is used as a basis for subsequent operations.

FIG. 9B illustrates a spinal canal extraction 902 based on image 901 (after conversion to black-and-white and some processing for pictorial clarity). Canal 912 is extracted using an initial segmentation by morphological region growing technique and subsequent fine 3D active surface segmentation. The curvature of extracted canal 912 typically represents the curve or curvature of the spine (see also below).

FIG. 9C illustrates detection of specific vertebrae and vertebrae bodies based on image 901 (after conversion to black-and-white and some processing for pictorial clarity).

Each vertebrae of the spine is detected, shown as corresponding dots 914, by initially applying a curved planar reformation (CPR) on the volume based on the extracted spine curve 912. Subsequently a generalized Hough transform (GHT) is applied to detect arbitrary shapes in an image undergoing geometric transformations and the description of the shape is encoded into a table where the entries are vectors pointing from the shape boundary to a reference point typically as center of mass (volume). During detection process, the gradient orientation is measured at each edge voxel of the resultant image yielding an index for an entry of the table. The positions pointed by all vectors under an entry are incremented in an accumulator array and the shape is determined by the highest peak in the accumulator array.

FIG. 9D illustrates identification of vertebrae based on image 901 (after conversion to black-and-white and some processing for pictorial clarity).

Extracted vertebra candidates are identified by registering the appearance models to the candidates and measuring the similarity of the detected objects to a given model. Identification is carried out in the original image, considering a small region of interest around the detected candidate to avoid computational effort. The maximal similarity determines the final position of the vertebrae 916.

FIG. 9e illustrates fine segmentation of vertebrae based on image 901 (after conversion to black-and-white and some processing for pictorial clarity).

The final segmentation 918 is carried out by adapting triangulated shape models of the individual vertebrae using a shape-constrained deformable models approach where an external force attracts the mesh triangles to image features while an internal constraint assures the model shape. Using a physical metaphor, the iterative procedure of mesh deformation is performed by minimizing an energy term.

### Spinal Canal

Since the spinal canal is an object having varying edges, namely sharp edges (with the spine), soft edges (with intervertebral disc) and no edges (with nerve roots), identifying and extracting the spinal canal is a challenging task. Several techniques to extract the canal have been suggested, for example, R. M. Rangayyan, H. J. Deglint, G. S. Boag, “Method for the automatic detection and segmentation of the spinal canal in CT images”, J. of Electronic Imaging, vol. 15, July 2006, pp. 033007-9, or T. Klinder, J. Ostermann, M. Ehm, A. Franz, R. Kneser, C. Lorenz, “Automated model-based vertebra detection, identification, and segmentation in CT images,” Medical Image Analysis, In Press.

The spinal canal is extracted by initial segmentation using morphological region growing technique which is followed by fine 3D active surface segmentation.

A seed point for the spinal canal segmentation is found using pattern recognition. The detection is carried out on a 2D image created by fusion of several axial slices that account for about 10 mm (or any interval depending on the scan parameters) at the superior part of the scan.

The seed point is the center of a hole which is detected by hole using circle detection Hough transform. Of the resulting circles, the most appropriate circle is chosen by morphological characteristics thereof and relation to bone segments on the fused image, namely, a hole at the mid posterior part of the image surrounded by bone.

To extract the initial region on the axial slice (where the seed is located), region growing segmentation, is performed. For example, based on similarity of pixel values together with JSEG texture values. If the initial 2D segmentation differs significantly from the hole (circle) detected above for seed point, the hole is taken as the initial 2D segmentation (although in many cases segmentation is smaller then the detected hole). Subsequently, the spinal canal is segmented by stepwise morphological region growing. At each step all neighborhood pixels within adaptive thresholds are considered where the candidates are then divided into connected components, and the most appropriate component (i.e. component with similar size and mean HU values) is added to the segmentation. At the end of the process leak detection is carried out by checking the size of the components at each step, and checking if there is a significant change of size indicating leakage.

Consequently a fine segmentation process based on a 3D discrete deformable model is performed. An initial boundary (a simplex mesh) is deformed under internal (shape-based) and external (image-based) constraints until an equilibrium is reached. To overcome weak edges a coarse-to-fine approach is used as (a) a low-resolution mesh is deformed until convergence, and then (b) the mesh is refined and deformed again but allowing only small deformation.

Mesh deformation is governed by a second order evolution equation, which can be rewritten for discrete meshes as follows:

$$m \frac{{\partial }^{2} {P}_{i}}{\partial {t}^{2}} = α \cdot Fint + β \cdot Fext$$

where α and β are global weights for the internal and the external forces respectively. This equation can be discretized in time t, using an explicit discretization scheme as follows:

Pᵢ^{t+1}=Pᵢ^{t}+α·Fint+β·Fext

The internal force imposes smoothness constraints on the polygonal mesh. External forces were computed along the normal of each vertex ‘on the fly’ to allow faster computation then separate processes. Both texture edge information and intensity information are combined for the computation of the external forces:

Fext=β₁F_{Texture}+β₂F_{Intensity}

The texture edge image was based on JSEG (J measure based segmentation) algorithm (see, for example, Y. Deng, B. Manjunath, “Unsupervised segmentation of color-texture regions in images and video,” Pattern Analysis and Machine Intelligence, IEEE Transactions on, vol. 23, 2001, pp. 800-810). The basic idea of the JSEG method was to separate the segmentation process into two stages: color quantization and spatial segmentation. In the first stage, quantization algorithm based on peer group filtering (PGF) and vector quantization reduces image gray to produce a class map. Based on the class map, spatial segmentation was performed based on pixel ‘J val’ (see, for example, Y. Wang, J Yang, Y. Chang, “Color-texture image segmentation by integrating directional operators into JSEG method,” Pattern Recognition Letters, vol. 27, December 2006, pp. 1983-1990).

The measure J is defined as:

$$J = \frac{{S}_{B}}{{S}_{W}} = \frac{{S}_{T} - {S}_{w}}{{S}_{W}}$$

Where S_{T} is the within class variance and S_{W} is the total variance. For each pixel, the corresponding J value was calculated over the local window (e.g. 5×5×5) centered on this pixel, thereby forming a J-image. To improve computation performance J values were computed only along the normal line for each vertex, the edge along the normal was found, and the texture force value was the product of the distance of the vertex to the edge, and the edge strength.

The intensity force is based on given range of intensity values. Assuming the vertex is within the object, the closest point along the normal to be outside the range is considered as the edge, and the force magnitude is defined as the distance to the edge.

The Mesh adaptation is carried out until no further deformation is observed (i.e. deformation step size is smaller than threshold) or certain number of iterations (e.g. 20) have passed, providing a spinal canal segmentation.

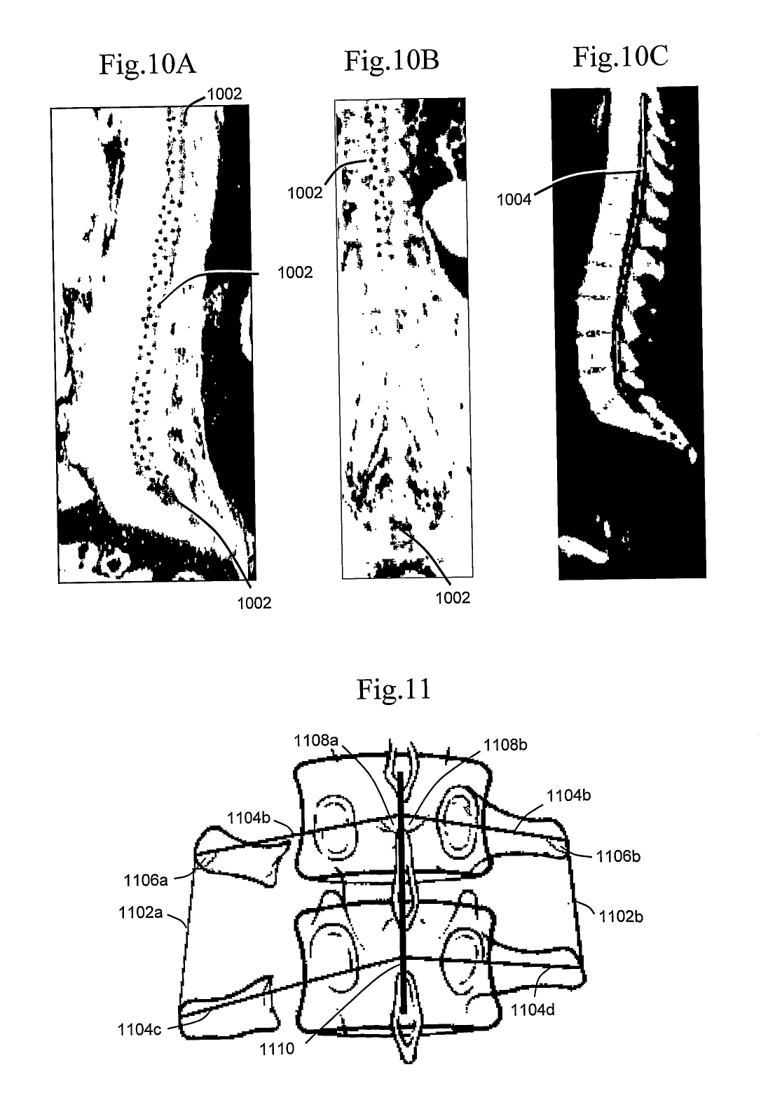

FIG. 10A illustrates a lateral view of a two-dimensional view of a volumetric CT study after initial segmentation of the spinal canal (with conversion to black-and-white and some processing for pictorial clarity). The segmentation spots 1002 are scattered about the curvature of the spinal cord.

FIG. 10B illustrates an anterior view of a two-dimensional view of a volumetric CT study after initial segmentation of the spinal canal (with conversion to black-and-white and some processing for pictorial clarity). The segmentation spots 1002 are scattered about the curvature of the spinal cord.

FIG. 10C illustrates a lateral view of a two-dimensional view of a volumetric CT study after centerline 1004 extraction based on the segmentation spots 1002 (with conversion to black-and-white and some processing for pictorial clarity).

In some embodiments, the spinal canal is extracted by segmentation followed by fast marching minimal path extraction to locate the centerline. The extraction may be summarized as: based on start and end points, a speed function to generate an arrival function is used and an optimizer which steps along the resultant arrival function perpendicular to the fast marching front is applied. The speed function is a distance from canal segmentation. The start (end) point is defined as the center of the segmentation at its topmost (bottom) axial slice and the optimizer is a Gradient Descent Optimizer.

The extracted spinal canal can be used for assessment of spine curvature and optionally spine torsion (see also below), where for a curve defined as a function r(t) the curvature κ at a given t is:

$$κ = \frac{\vert \dot{r} \times \ddot{r} \vert}{{\vert \dot{r} \vert}^{3}}$$

where \dot{r} and \ddot{r} are first and second derivatives of r(t), and the torsion (twisting) τ is defines as:

$$τ = \frac{det ( \dot{r} , \ddot{r} , {r}^{⃛} )}{{\vert \dot{r} \times \ddot{r} \vert}^{2}} = \frac{\vert \dot{r} \times \ddot{r} \vert \cdot {r}^{⃛}}{{\vert \dot{r} \times \ddot{r} \vert}^{2}}$$

### Muscles

As an example of muscles segmentation a method for an atlas-based automated segmentation of spine muscles is briefly overviewed below for volumes of muscles such as Psoas Major, Quadratus Lumborum or Erector Spinea.

Based on manually segmenting muscles of interest on axial MR or CT images of the lumbar region an average population atlas is generated. The spine muscles are then segmented by affine registration followed by non-rigid registration to allow segmentation of the muscles using geodesic active contours using propagation of a probabilistic atlas derived from the existing MR/CT dataset atlases. In order to approximate the voxel resolution of the image dataset a spline interpolation on the images is applied resulting in uniform resolution.

Some more details on the methods of affine registration, non-rigid registration, classification, atlas creation and propagation in segmentation are provided further below.

### Vertebra Analysis

A reference is made to Table-2 above and corresponding FIG. 4.

Each vertebra is detected and segmented as described above, and the vertebra features (e.g. vertebra's body, endplates, arc, processes, lamina, and pedicle) are identified and examined for properties such as surface texture, surface inhomogeneity (roughness or creases of surface), cracks or Schmorl's nodes by using image processing techniques.

Each identified and segmented vertebra or, optionally, particular vertebrae (such as representative or suspected vertebra from earlier studies), is analyzed as described below.

The listed analyses are non-limiting representative ones, wherein in some embodiments the listed analyses are performed fully or partially and wherein in some embodiments, at least optionally, other analyses are performed.

### Endplates (Discal Surface) Lateral and Anterior Posterior Diameters

The contour of a vertebra's body endplate (top and bottom of each vertebra body) is identified. For each endplate contour a bounding box is constructed. The anterior posterior size of the box represents the endplate's anterior posterior diameter while the lateral size of the box represents the endplate's lateral diameter.

### Endplate Area and Effective Diameter

The cross section of the vertebra's body endplate (top and bottom of each vertebra body) is calculated, and based on the endplate's cross section the effective diameter is calculated, namely the diameter of a circle with the same area.

### Vertebra Body Height

The vertebra body height is calculated, using distance between vertebra body's endplates employing image processing techniques such as Hausdorff distance, mean points distance, mean or median of distances along the vertebra body midline, or average distance between distinctive points such as most anterior points and most posterior points on each endplate.

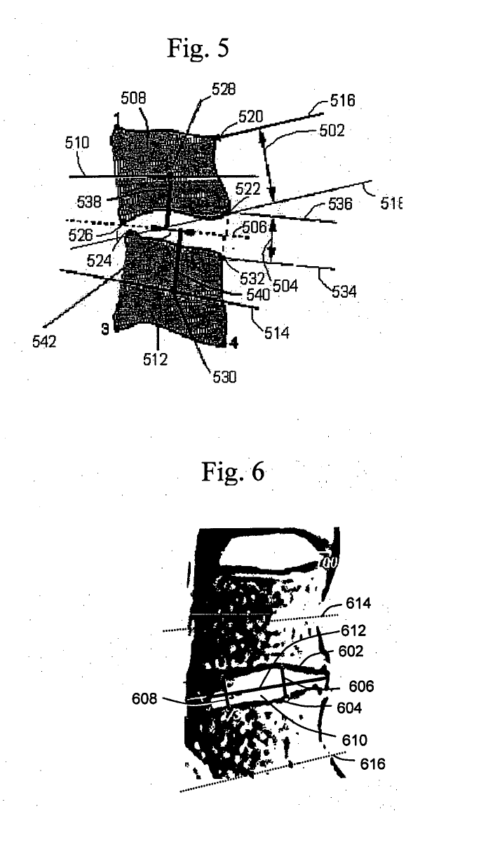

FIG. 5 schematically illustrates how a height of a vertebra is measured, according to exemplary embodiments of the invention.

Considering two adjacent vertebrae, top vertebra 508 and bottom vertebra 512, the center points 528 and 530 of each vertebra, respectively, are determined, such as by center of mass (volume), and midplanes 510 and 514, respectively, are constructed by equally dividing each mass.

A bisector plane (or line) 506 dividing equally the angle between midplanes 510 and 514, or parallel thereof to that matter, is constructed. The anterior height 502 of top vertebra 508, as an example, is determined by the distance between (a) a line 518 (t-line) from lower anterior end point 522 of top vertebra 508 to upper posterior end point 524 of bottom vertebra 512 and (b) a line 516 passing through upper anterior end point 520 of top vertebra 508 parallel to line t-line 518.

This method for determining a vertebra height above is provided as an example, and other methods and/or combinations thereof may be used as well (e.g. averaging posterior and anterior heights to provide a representative height).

### Vertebra Body Volume

The vertebra body volume is calculated employing image processing such as computational geometry on segmented vertebra body.

### Coronal and Sagittal Beveling of Vertebra

Endplates of a vertebra is detected and each endplate is fitted with a 2D plane and the angle between the planes along each direction, or along the vertebra body middle cut along coronal and sagittal axes is calculated.

### Density of Vertebra's Body Cortical Bone

The density of the cortical bone component of a vertebral body is calculated by identifying cortical bone such as by pixel classification or pattern recognition, segmentation of cortical bone and calculation of bone density from volume intensity such as by mean or median of pixels values of the segmented element.

### Thickness of Vertebra's Body Cortical Bone

The cortical bone of a vertebra body is identified and segmented and the thickness is calculated based on the segmentation.

### Density of Vertebra's Body Trabecular Bone

The bone component of vertebral body is identified and segmented and the volume is calculated such as by mean or median of pixels values of the segmented element.

### Directional Characteristics of Trabecular Bone

The trabecular bone is identified and segmented and directional characteristics of the trabecular bone are calculated such as by volume texture filters, or pixel classification followed by directional run length encoding of the body or line crossing along directional planes.

### Density of Vertebra's Body Subchondral Bone

The subchondral bone of a vertebra is identified and segmented and the bone density is calculated from the volume intensity such as by mean or median of pixels values of the segmented element.

### Thickness of Vertebra's Body Subchondral Bone

The subchondral bone component of a vertebral is identified and segmented and the thickness of the subchondral bone is calculated.

### Vertebra's Processes Morphology and Configuration

Each vertebra process is identified using image processing such as pattern recognition techniques or model fitting and for each process the apex and the connection point to the neural arc are identified by image processing such as using feature pattern recognition, model features identification, or correlation. Having identified each process apex and connection points, the following characteristics are calculated.

(i) Articular, transverse and spinous processes lengths: Given the process' apex and arc connection point, the process length is taken as the 3D distance between the two points.

(ii) Articular, transverse and spinous processes width: Given the process' apex and arc connection point, cross sectional cuts of the processes on planes perpendicular to the centerline equidistance along the line are created, and the processes contours are identified based on subsequent processes segmentation. For each cross sectional cut contour the width of the processes is obtained and for all cross sectional cuts the minimal, average and maximal width of the processes are calculated as defined on projection of the processes.

(iii) Articular and transverse processes angle to the vertebra anterior posterior mid sagittal plane: The anterior posterior mid plane is found in a vertebra using methods such as (1) fitting the vertebra to a vertebra model or to image from a repository or other external sources and identifying at the midline through the model or atlas, or (2) using the mid plane of vertebra body to be calculated by image processing or geometrical methods, or (3) using the mid plane of the whole vertebra calculated by image processing or geometrical methods. Next the angle between the line defined by the process' apex and arc connection point and the vertebra mid plane is calculated such as by geometrical methods.

(iv) Superior/Posterior inter-facet width: Based on the apex points of the two Superior or Posterior articular process' apex points of each vertebra, the 3D distance between the two points is calculated.

(v) Left/Right Inter facet height: Based on the apex points of the two left or right articular process' apex points of a vertebra the 3D distance between the two points is calculated.

(vi) Distance between transverse processes apexes: based on the apex points of the two transverse process' apex points of a vertebra the 3D distance between the two points is calculated.

### Spondylolysis Identification

A fracture in the pars interarticularis of a vertebra is identified, for example, by detecting small disconnection of bone along the vertebra's arc using pattern recognition techniques (e.g. ridge identification) or morphological discontinuity of the vertebra (the arc is not closed).

### Schmorl's Nodes

Schmorl's nodes are identified by detecting endplate irregularities, such as holes in vertebrae end plates, density and/or texture differences, evaluation or detection of disc material (such as by using material classification, texture, shape analysis) in the endplate and inside the vertebral body and by subsequent analysis of the size (area), shape, and location of Schmorl's node on the endplate. (Schmorl's nodes are protrusions of the cartilage of the intervertebral disc through the vertebral body endplate and into the adjacent vertebra).

### Bone Osteophytes

Irregularities on bone surfaces indicating abnormal bone growth are identified. Based on segmentation of the spine, the surface of the bones are extracted. An osteophyte is identified by locating sharp irregular changes of the surface (e.g. calculating surface curvature and analysis), or by comparing the surface to a bone model or repository image and identifying respective large deviations of the bone. Connectivity analysis is optionally conducted for connecting adjacent irregularities, ascribing every cluster of irregularities as a single bone osteophyte.

### Pedicles Characteristics

A pedicle is defined here as the arch component between the origin of the arch (the attachment of the arch to the vertebra body) and the transverse process (as define by extension of the midline of the transverse process to the arch). The following characteristics of a pedicle are calculated.

(i) Pedicles length: On axial cross section of the vertebra a pedicle length is calculated as either as the length of the straight line from the origin of the arch to the transverse process, or as the length along a geodesic curve along the arch midline between the transverse process to the vertebra body. On a cross section image, the center point/lateral end point/medial end point of the border between the arch and the body is one end point of the line, and the center point/lateral end point/medial end point of the extension of the transverse process midline to the arch is the other endpoint, and the length is calculated as either 3D distance between the two points or a geodesic line along the arch (pedicle) mid line.

(ii) Pedicles curvature: The ratio between (a) the distance from the origin of the arch to the transverse process, and (b) the length along a geodesic curve along the arch midline between the transverse process to the vertebra body.

(iii) Pedicles width: Cross sectional cuts of the pedicles on planes perpendicular to the centerline equidistance along the arc line midline from the transverse process to the vertebra body are constructed, and the pedicle's contours are detected based on the pedicle segmentation. The width of the pedicle is calculated for each cross sectional cut contour, and for all cross sectional cuts the minimal, average and maximal width of the pedicle as defined on projection of the pedicle are calculated.

(iv) Pedicles height: Cross sectional cuts of the pedicles on planes perpendicular to the centerline equidistance along arc line midline from the transverse process to the vertebra body are constructed, and the pedicle's contours are detected based on the pedicle segmentation. The height of the pedicle is calculated and for all cross sectional cuts the minimal, average and maximal height of the pedicle as defined on axial of the pedicle is calculated.

(v) Pedicles density: A pedicle density is calculated from volume intensity, such as by mean or median of pixels values of the segmented pedicle.

### Lamina Characteristics

A lamina is defined here as the arch component between the transverse process (as defined by extension of the midline of the transverse proves to the arch) and the spinous process (as defined by extension of the midline of the spinous process to the arch). The following characteristics of a lamina are calculated.

(i) Lamina length: On axial cross section of the vertebra the lamina length is calculated as either the length of the straight line from the transverse process to the spinous process, or the length along a geodesic curve along the arch midline between the transverse process to spinous process. On the cross section image, the center point/lateral end point/medial end point of the extension of the transverse process midline to the arch is one end point of the line, and the center point/lateral end point/medial end point of the extension of the spinous process midline to the arch is the other endpoint and the length is calculated as either 3D distance between the two points or a geodesic line along the arch (pedicle) mid line.

(ii) Lamina curvature: The ratio between (a) the distance from the transverse process to the spinous process, and (b) the length along a geodesic curve along the arch midline between the transverse process to the spinous process.

(iii) Lamina width: Cross sectional cuts of the lamina on planes perpendicular to the centerline equidistance along the line are constructed along the arc line midline from the transverse process to the spinous process, and the lamina's contours are detected based on the lamina segmentation. The width of the lamina is calculated for each cross sectional cut contour, and for all cross sectional cuts the minimal, average and maximal width of the lamina as defined on projection of the lamina are calculated.

Lamina height: Cross sectional cuts of the lamina on planes perpendicular to the centerline equidistance are constructed along the arc line midline from the transverse process to the spinous process, and the Lamina's contours are detected based on the lamina segmentation. The height of the lamina is calculated for each cross sectional cut contour, and for all cross sectional cuts the minimal, average and maximal height of the lamina as defined on projection of the lamina are calculated.

(v) Laminas density: A lamina density is calculated from volume intensity, such as by mean or median of pixels values of the segmented lamina.

### Width of the Epiphyseal Ring

The epiphyseal ring is identified using image processing techniques and based on segmenting the epiphyseal ring the anterior, posterior, left and right width of the ring are calculated. (Apophysis at the circumference of the upper and lower margin of the vertebral body.)

### Spinal Canal and Spinal Cord Analysis

The spinal canal and spinal cord are identified and segmented by image processing techniques. For example, (1) using body landmarks such as spine to identify initial point in the spinal canal followed by segmentation techniques such region growing, active shape, (2) level sets, graph cuts, or volumetric watershed transform based on image intensities, texture, and prior data based on the bony boundaries, (3) model based identification and fitting of the spine, or (4) registration and segmentation of the canal and spinal cord based on repository images. Using image processing, such as skeletonization, shortest path using graphs methods, fast marching methods or medial line, the spinal canal and spinal cord centerline are calculated.

Based on the centerlines, the following characteristics of the spine are calculated as described below. The listed calculations are non-limiting representative ones, wherein in some embodiments the listed calculations are performed fully or partially and wherein in some embodiments, at least optionally, other calculations are performed.

### Spinal Canal Cross Sectional Area and Effective Diameter Along the Canal

Cross sectional cuts of the canal on planes perpendicular to the centerline equidistance along the line are constructed, and the canal's contours are detected based on the canal segmentation. Based on a cross sectional cut contour, each contour area is calculated using geometrical methods. According to the cross sectionals area the effective diameter is calculated, namely the diameter of a circle with the same area.

### Spinal Canal Lateral and Anterior Posterior Diameter Along the Canal

Cross sectional cuts of the canal on planes perpendicular to the centerline equidistance along the line are constructed, and the canal's contours are detected based on the canal segmentation. For each cross sectional cut contour a bounding box is constructed. The anterior posterior size of the box represents the canal's anterior posterior diameter while the lateral size of the box represents the canal's lateral diameter.

### Spinal Cord Cross Sectional Area and Effective Diameter Along the Canal

Cross sectional cuts of the spinal cord on planes perpendicular to the centerline equidistance along the line are constructed, and spinal cord's contours are detected based on the spinal cord segmentation. For each cross sectional cut contour the contour area is calculated using geometrical methods. According to the cross sectional area the effective diameter is calculated, namely the diameter of a circle with the same area.

### Spinal Cord Lateral and Anterior Posterior Diameter Along the Canal

Cross sectional cuts of the spinal cord are constructed on planes perpendicular to the centerline equidistance along the line, and the spinal cord's contours are detected based on the spinal cord segmentation. For each cross sectional cut contour a bounding box is constructed. The anterior posterior size of the box represents the spinal cord's anterior posterior diameter while the lateral size of the box represents the spinal cord's lateral diameter.

### Density of the Canal

The canal density is calculated from volume intensity, such as by mean or median of pixels values of the segmented canal.

### Density of the Spinal Cord

The spinal cord density is calculated from volume intensity, such as by mean or median of pixels values of the segmented cord.

### Partial Cross Sectional Area for Fat, Air, Ligament and the Spinal Cord

Cross sectional cuts of the canal cord on planes perpendicular to the centerline equidistance along the line are constructed, and spinal cord's contours are detected based on the spinal cord segmentation. The pixels in each cross sectional cut contour of the canal are classified to one of the following categories: spinal cord, fat, air, ligament and intervertebral disc gel by using classification methods taking into account data such as pixel intensity values, pixel morphological location, pixel neighborhood, and pixel's features such as texture. Optionally or additionally, the classification can be carried out using machine based classification methods such as Gaussian mixture models, neural networks or support vector machines.

### Intervertebral Disc Analysis

Based on the vertebrae identified as describe above, the intervertebral discs located between two vertebra bodies are identified.

Using image processing techniques a disc is segmented and pixels are separated into nucleus pulposus, and annulus fibrosus regions. Subsequently, the disc main axes and orientation are detected using either the disc segmentation, the top and bottom vertebra end plate, or using the spinal canal centerline cut planes. Based on the disc axes, the disc's axial mid plane and the disc's sagittal and coronal mid planes are detected.

The following characteristics of the discs are calculated as described below. The listed calculations are non-limiting representative ones, wherein in some embodiments the listed calculations are performed fully or partially and wherein in some embodiments, at least optionally, other calculations are performed.

### Example of Intervertebral Disc Segmentation in CT Scans

Given vertebrae bodies' segmentation and identification, the intervertebral disc segmentation is carried out by two consecutive steps, namely, (i) initial disc segmentation and (ii) final disc segmentation, as follows.

(i) Iteratively applying morphological dilation using a 1D structuring element along the direction from a vertebra body center to the next vertebra body center until the two vertebrae are joined.

(ii) Applying 3D active contours (adapting triangulated shape models of the disc) using a shape-constrained deformable models approach where an external force attracts the mesh triangles to image features while an internal constraint assures the model shape while assuming that disc edges are defined by CT values and by JSEG texture similarity. Using a physical metaphor, the iterative procedure of mesh deformation is performed by minimizing an energy term.

### Anterior/Posterior Disc Height and Disc Height

Cross sectional cut of the disc are constructed on the disc's sagittal mid-plane, and the disc's contour based on its segmentation are detected. Top and bottom lines are fitted on the disc contour, representing top and bottom cross section regions. On each line (top and bottom) anterior and posterior points are identified and the 2D distances of the anterior top and bottom points and the posterior top and bottom points are calculated, wherein Anterior/Posterior Disc height is the average distance thereof.

The disc height is calculated using distance methods such as distance between neighboring top and bottom vertebrae endplates, mean or median of distances along the vertebra body midline, or average distance between feature points such as most anterior points and most posterior points on each endplate.

FIG. 5 that schematically illustrates how a height of an intervertebral disc is measured, according to exemplary embodiments of the invention, is referenced again.

Considering two adjacent vertebrae, top vertebra 508 and bottom vertebra 512, the center points 528 and 530 of each vertebra, respectively, are determined, such as by center of mass (volume), and midplanes 510 and 514, respectively, are constructed by equally dividing each mass.

A bisector plane (or line) 506 dividing equally the angle between midplanes 510 and 514, or parallel thereof to that matter, is constructed. The anterior disc height is evaluated as the sum of (a) a perpendicular line to bisector plane 506 from lower anterior end point 522 of top vertebra 508 and (b) a perpendicular line to bisector plane 506 from upper anterior end point 532 of bottom vertebra 512 (or equally as the distance between lines 536 and 534 parallel to bisector plane 506 and passing through lower anterior end point 522 of top vertebra 508 upper anterior end point 532 of top bottom 512, respectively).

FIG. 6 schematically illustrates an alternative exemplary method for measuring the height of an intervertebral disc based on an image such as X-ray or CT (after some processing and conversion to black and white rendering), according to exemplary embodiments of the invention.

Considering disc 610, midline 614 of superior vertebra above disc 610 and midline 616 of inferior vertebra below disc 610 are determined and a bisector 612 of the angle between midlines 614 and 616 is constructed within disc 610. At ⅓ distance from anterior and posterior boundaries of disc 610 perpendicular lines 606 and 608 (respectively) are constructed to upper and lower boundaries 602 and 604 of disc 610 with superior and inferior vertebrae, respectively. The average (e.g. arithmetic average) of perpendicular lines 606 and 608 is determined as an estimate or measurement of the height of disc 610.

The methods for determining a disc height above are provided as an example, and other methods and/or combinations thereof may be used as well (e.g. averaging posterior and anterior heights to provide a representative height).

### Cross Sectional Area and Effective Diameter of Intervertebral Disc

Cross sectional cut of the disc is constructed on the disc's axial mid-plane, and the disc's contour is detected based on segmentation. The contour area is calculated using geometrical methods and according to the cross sectional area the effective diameter is calculated, namely the diameter of a circle with the same area.

### Lateral and Anterior Posterior Diameter of Intervertebral Disc

Cross sectional cut of the disc is constructed on the disc's axial mid-plane, and the disc's contour is detected based on the disc segmentation. For each cross sectional contour a bounding box is constructed. The anterior posterior size of the box represents the disc's anterior posterior diameter while the lateral size of the box represents the disc's lateral diameter.

### Disc Density

The spinal disc density is calculated from volume intensity, such as by mean or median of pixels values of the segmented disc.

### Disc Water Content

The volume of water inside the disc is calculated using imaging and/or image processing techniques capable of material separation.

### Nucleus Pulposus Cross Sectional Area (and Ratio to Entire Disc Area)

Cross sectional cut of the disc is constructed on the disc's axial mid-plane, and the disc's contour is detected based on the disc segmentation. The contour area and the nucleus area are calculated, such as by geometrical methods, and the nucleus ratio to the disc area is computed.

### Nucleus Pulposus Density

The nucleus pulposus density is calculated from volume intensity, such as by mean or median of pixels values of the segmented nucleus pulposus.

### Nucleus Pulposus Water Content

The volume of water inside the Nucleus pulposus is calculated using imaging and/or image processing techniques capable of material separation.

### Annulus Fibrosus Cross Sectional Area

Cross sectional cut of the disc is constructed on the disc's axial mid-plane, and the disc's contour is detected based on the disc segmentation. The contour area and the annulus fibrosus cross sectional area (the part of the disk devoid of the nucleus) are calculated, such as by geometrical methods, and the annulus fibrosus ratio to the disc area is computed.

### Annulus Fibrosus Anterior, Posterior and Lateral Widths

Cross sectional cut of the disc is constructed on the disc's axial mid-plane, and the disc's contour is detected based on the disc segmentation. The area identified with the following lines: disc mid anterior posterior line and disc mid lateral line is cut off and on the disc mid anterior posterior line two parts of annulus are detected—anterior part and posterior part. For each of the two parts (anterior and posterior), the length of the cut for each part on the line intersecting disc center defines the annulus anterior and posterior widths. Lateral widths are obtained by carrying out similar procedure on the lateral line.

### Annulus Fibrosus Density

The annulus fibrous density is calculated from volume intensity, such as by mean or median of pixels values of the segmented annulus fibrosus.

Vacuum within Disc

Vacuum inside intervertebral discs is identified using tissue classification based on materials density as vacuum or air have very low density relative to other constituents of the spine (e.g. water, fat, muscle, bone), and can be easily differentiated from the disc. The identified disc vacuum is segmented and the vacuum size (volume, cross sectional area) and relative location inside the disc (orientation and distance from the center) are calculated.

### Muscles Analysis

Using the previously identified vertebrae, the muscles of the spine and trunk are identified and segmented. Based on a muscle or the bones the muscle attached to, the origin and insertion locations of the muscle are detected. Subsequently the muscle medial line along the origin and insertion locations of the muscle (e.g. from center of muscle origin to center of muscle insertion) is detected using methods such as medial line extraction techniques, skeletonization or path finding techniques.

The following characteristics of the muscles are calculated as described below. The listed calculations are non-limiting representative ones, wherein in some embodiments the listed calculations are performed fully or partially and wherein in some embodiments, at least optionally, other calculations are performed.

### Muscle Direct (Line) Length

3D distance from center of origin to center of insertion using distance methods such as Hausdorff distance.

### Muscle Length Along the Medial Line

The distance from the center of origin to center of insertion along the muscle medial line, actually the medial line length.

### Muscle Curvature

The ratio between the muscle length along the medial line thereof and the muscle's direct length.

### Muscle Lateral and Anterior Posterior Width Along the Length Thereof

Cross sectional cuts of the muscle are constructed on planes perpendicular to the medial line equidistance along the medial line, and the muscle's contours are detected based on segmentation. For each cross sectional contour a bounding box is constructed. The anterior posterior size of the box represents the muscle's anterior posterior (diameter) while the lateral size of the box represents the muscle's lateral width (diameter).

### Muscle Cross Sectional Area and Effective Diameter Along the Length Thereof

Cross sectional cuts of the muscle are constructed on planes perpendicular to the medial line equidistance along the medial line, and the muscle's contours are detected based on segmentation. For each cross sectional cut contour the contour area is calculated using, for example, geometrical methods, and based on contour area the effective diameter is calculated, namely the diameter of a circle with the same area.

### Muscle Density

The muscle's density is calculated from volume intensity, such as by mean or median of pixels values of the segmented muscle.

### Muscle Fat Content (Ratio of Fat Volume to Muscle Volume)

The largest cross section of the muscle or an axial cut along a specific plane of a vertebra (e.g. L4 top endplate, or L3 vertebra body axial mid plane) is regarded as the whole (or substantially the whole) muscle. The pixels of the muscle are classified to fat or muscle regions using methods such as Gaussian mixture model or support vector machines, and the ratio of the fat pixels volume to the muscle volume is determined.

### Muscle Origin and Insertions Area

For each muscle origin and insertion locations the attachment area—area of contact between bone and muscle—is determined using, for example, geometrical methods (e.g. surface of the attachment pixels).

### Ligaments Analysis

Using the previously found elements (such as bones, bones elements, muscles), the spine ligaments are identified and segmented. For a ligament the insertion locations are determined, for example, based on the area of contact between bone and ligament or the bones the ligament is attached to. Subsequently the ligament's medial line is calculated from proximal to distal insertion points using methods such as medial line extraction techniques, skeletonization, or path finding techniques.

The following characteristics of the ligaments are calculated as described below. The listed calculations are non-limiting representative ones, wherein in some embodiments the listed calculations are performed fully or partially and wherein in some embodiments, at least optionally, other calculations are performed.

In some embodiments, ligaments segmentation follow similar techniques as muscle segmentation. For example, using a database of CT or MR images with manually segmented ligaments to create an atlas of the ligament, registrations of patient scan to the atlases using affine registration followed by non-rigid registration and eventually segmentation of the ligaments using geodesic active contours through propagation of a probabilistic atlas derived from the existing MR/CT dataset atlases.

### Ligaments Direct (Line) Length

3D distance from proximal to distal insertion points using distance methods such as Hausdorff distance.

### Ligaments Length Along its Medial Line

The distance from proximal to distal insertion points along the ligament's medial line, actually the direct line length.

### Ligament's Curvature

The ratio between the ligament's length along the medial line thereof and the ligament's direct length.

### Ligament's Cross Sectional Area and Effective Diameter Along the Length Thereof

Cross sectional cuts of the ligament on planes perpendicular to the medial line equidistance along the line are constructed, and the ligament's contours are detected based on segmentation. For each cross sectional cut contour the contour area is calculated using, for example, geometrical methods, and based on contour area the effective diameter is calculated, namely the diameter of a circle with the same area.

### Ligaments Density

The ligament density is calculated from volume intensity, such as by mean or median of pixels values of the segmented ligament.

### Ligaments Proximal and Distal Insertion Area and Effective Diameter Thereof

The attachment area of the proximal and distal insertion locations of a ligament—area of contact between bone and ligament—is detected by using, for example, geometrical methods (e.g. surface of the attachment pixels). Based on the cross sectional area, the effective diameter is calculated, namely, the diameter of a circle with the same area.

### Syndesmophytes

Syndesmophytes can be detected by identifying calcification (pixels with high CT values) inside the ligament, using classification methods such as threshold, Gaussian mixture models or radial basis function. (Syndesmophytes are bony growth originating inside a ligament.)

### Morphological Analysis

The morphology of the spine and parts (elements) thereof is analyzed to obtain quantitatively characteristic and relations between the spine's parts.

The term ‘morphology’ (and derivative thereof) refers also to anatomical aspects of the spine and parts thereof.

The morphological analysis is carried out by employing image processing and computational geometry techniques and is performed automatically or semi-automatically by involving some user interaction.

The analyses listed below are non-limiting representative ones and are generally, without limiting, based on analyses and/or calculations described above, at least partially. In some embodiments the listed analyses are performed fully or partially and in some embodiments, at least optionally, other analyses are performed.

### Global Morphology

Some of the spine characteristics are global in the sense that they span over the entire spine, at least approximately, or represent a specific configuration or property of the spine.

Following are some global characteristics based on features or elements such as vertebrae endplates, center of vertebrae bodies or anterior and posterior corners of vertebra body on its mid-plane.

(i) Overhang: Horizontal offset between the midpoint of the sacral plate and the femoral heads axis.

(ii) T9 projection: Horizontal distance between inferior anterior corner of a vertebra body on the mid-plane thereof and the line between the femoral head.

(iii) Curvature amplitude: The maximum distance of the vertebral anterior walls from the spine best-fitting straight line, in kyphosis and lordosis. The amplitude of the curvature is expressed as a percentage of the length of the sacral plate

(iv) Spine curviness: The ratio of the spine curvature (sum of distance between consecutive centers of vertebra bodies from C1 to L5) to spine height (distance between the center of vertebra body of C1 and vertebra body of L5).

(v) Lumbar lordosis angle: Using methods such as Cobb, or Harrison methods.

(vi) Thoracic kyphosis angle: Using methods such as Cobb, or Harrison methods.

(vii) Sacral slope: The angle between the horizontal plane and the sacral plate.

(viii) Sacral inclination

(ix) Pelvic tilt: The angle between the vertical and the line through the midpoint of the sacral plate to femoral heads axis (retroversion is then measured as a pelvic tilt increase, anterversion as a pelvic tilt decreases).

(x) Ankylosing spondylitis (sacrum-ileum ossification): Irregularities on bone surfaces of the sacrum indicating abnormal bone growth (see osteophytes above). Osteophytes are checked whether they cause integration of sacrum and ileum.

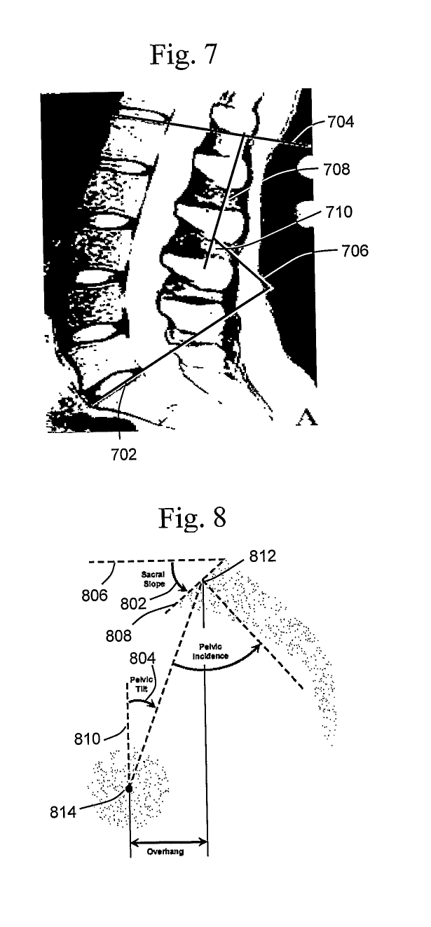

FIG. 7 schematically illustrates how a Cobb angle 610 is measured, according to exemplary embodiments of the invention.

Two lines 702 and 704 are constructed through the superior endplates of S1 and L1, respectively. Perpendicular lines 706 and 708 are drawn and extended from lines 702 and 704, respectively, and the angle 710 (marked as Θ) at the intersection thereof is denoted as the Cobb angle.

FIG. 8 schematically illustrates how a sacral slope 802 and a sacral tilt 804 are measured, according to exemplary embodiments of the invention.

Sacral slope 802 is defined as the angle between the horizontal line (or plane) 806 and the sacral plate 808

Pelvic tilt 804 defined by the angle between the vertical 810 and the line through the midpoint 812 of the sacral plate 808 to femoral heads axis.

### Local Morphology

Following are some local characteristics of relative morphology between elements of the spine (e.g. position, orientation).

(i) Relative vertebra body's center location: Location of vertebra body's center relative to neighboring vertebra bodies' centers. Based on vertebrae's body centers of the vertebrae, the distance of vertebra body's center from a curve based on neighboring vertebra bodies' centers is detected.

One exemplary implementation is the distance of vertebra body's center from a 3D line defined by the neighboring upper and lower vertebrae bodies' centers. Another exemplary implementation is the distance from general curve (e.g. 3D line, 3D polynomial curve of order N) defined by vertebrae's body centers.

(ii) Relative location of vertebra body's lateral/anterior/posterior border to neighboring vertebra bodies' lateral/anterior/posterior borders: Based on vertebra bodies such as lateral or anterior or posterior border, the distance of vertebra body's border such as lateral or anterior or posterior border from a curve based on neighboring vertebra bodies' border such as lateral or anterior or posterior border is determined.

An exemplary implementation is the mean distance of vertebra body's border from a 3D line or plane defined by the neighboring upper and lower vertebrae bodies' border. Another exemplary implementation is the distance from general line curve or surface (e.g. 3D line or plane, 3D polynomial curve of surface of order N) defined by vertebrae body borders.

(iii) Relative intervertebral disc's center location: Location of intervertebral disc's center relative to neighboring vertebra bodies' centers. Based on vertebra bodies centers of the vertebrae, the distance of intervertebral disc's from a curve based on neighboring vertebra bodies' centers is determined. An exemplary implementation is a distance of intervertebral disc's center from a 3D line defined by the neighboring upper and lower vertebrae bodies' centers. Another exemplary implementation is the distance from general curve (e.g. 3D line, 3D polynomial curve of order N) defined by vertebrae body centers.

(iv) Relative location of intervertebral disc's lateral/anterior/posterior border to neighboring vertebra bodies' lateral/anterior/posterior borders: based on vertebra bodies border such as lateral or anterior or posterior border, the distance of intervertebral disc's lateral border such as anterior or posterior border from a curve based on neighboring vertebrae body border. An exemplary implementation is the mean distance of intervertebral disc's border from a 3D line or plane defined by the neighboring upper and lower vertebrae body border. Another exemplary implementation is the distance from general line curve or surface (e.g. 3D line or plane, 3D polynomial curve of surface of order N) defined by vertebrae bodies lateral/anterior/posterior borders.

(v) Relative intervertebral disc's nucleus center location: Location of intervertebral disc's nucleus center relative to neighboring vertebrae body centers. Based on vertebrae body centers, the distance of intervertebral disc's nucleus center from a curve based on neighboring vertebrae body centers. An exemplary implementation is the distance of intervertebral disc's nucleus center from a 3D line defined by the neighboring upper and lower vertebrae body centers. Another exemplary implementation is the distance from general curve (e.g. 3D line, 3D polynomial curve of order N) defined by vertebrae body centers.

(vi) Muscles' attachments characteristics: Muscles attachment (origin or insertion) exhibits several morphological characteristics such as location and relative location, size, or shape. A muscle attachment is the surface where a muscle connects to the bone wherein the surface can be defined by image processing techniques such as the intersection surface of muscle and bone. Based on the muscle attachment surface the surface area, location and shape is or can be determined using image processing or computational geometry techniques.

(vii) Ligaments attachments characteristics: Ligaments attachment exhibits several morphological characteristics such as location and relative location, size, or shape. A ligaments attachment is the surface where the ligament connects to the bone wherein the surface is or can be defined by image processing techniques such as the intersection surface of muscle and bone Based on the ligament attachment surface the surface area, location and shape is or can be determined using image processing or computational geometry techniques.

FIG. 5 that schematically illustrates how a height of an intervertebral disc and disc center displacement are measured, according to exemplary embodiments of the invention, is referenced again.

Considering two adjacent vertebrae, top vertebra 508 and bottom vertebra 512, the center points 528 and 530 of each vertebra, respectively, are determined, such as by center of mass (volume), and midplanes 510 and 514, respectively, are constructed by equally dividing each mass, and a bisector plane (or line) 506 dividing equally the angle between midplanes 510 and 514, or parallel thereof to that matter, is constructed. The distance between vertebrae 508 and 512 is evaluated as the sum of (a) a perpendicular line 538 to bisector plane 506 from center point 528 of vertebra 508 and (b) a perpendicular line 540 to bisector line (or plane) 506 from center point 530 of vertebra 512.

The distance 542 on bisector 506 between the intersections of perpendicular lines 538 and 540 is considered as the disc center displacement.

### Motion Segment Morphology

A spinal motion segment, also known as the functional spinal unit, comprises two adjacent vertebrae and three joints (two posterior facet joints and the intervertebral disc) with ligaments between the joints. Any pair of adjacent vertebrae (except the fused sacral and coccygeal vertebrae) constitute a motion segment and enables movement of the spine.

The following morphological characteristics of the motion segment are calculated as described below. The listed calculations are non-limiting representative ones, wherein in some embodiments the listed calculations are performed fully or partially and wherein in some embodiments, at least optionally, other calculations are performed.

### Motion Segment Height

A motion segment height is determined by calculating a distance. For example, distance between superior vertebra superior endplate and inferior vertebra inferior endplate, mean or median of distances along the vertebra body midline, average distance between distinct points such as most anterior points and most posterior points on each endplate.

### Segment Height to Disc Height Ratio

The ratio between a segment height and disc height (see above).

### Ligamentum Flavum Length to Segment Height

The ratio between a length of ligamentum flavum of a segment and the segment height (see above).

### Motion Segment Ratio of Vertebra Bodies Area

Based on calculation of the area of the superior vertebra superior endplate and area of inferior vertebra inferior endplate, the ratio between the respective areas (superior to inferior) is computed.

### Orientation of Anterior/Posterior/Left/Right Segment Border

The orientation is defined as an angle between (a) a line constructed between a point on the superior vertebra superior endplate to a point on the inferior vertebra inferior endplate, and (b) a line between either the centers of the two vertebra bodies, or the plane defined by the inferior vertebra inferior endplate. Each endplate is one of anterior or posterior or left or right endplate.

### Left/Right Zygapophyseal Joints Distance Ratio

The ratio of the distance between the zygapophyseal joints of same side of a vertebra.

### The Left/Right Angle

The left or right angle is defined as the difference of orientations of the respective left or right zygapophyseal joints of the superior and inferior vertebrae.

### Anatomical/Morphological Assessment

A spine of a patient is assessed for anomalies such as asymmetry, irregularity, or deformities or other conditions which may affect the spine's morphology and biomechanics. The assessment is based on (a) extracted and (optionally) normalized spine elements as described above and/or a model thereof (such as represented by model 100 of FIG. 1), and (b) analysis and evaluations of a spine and elements thereof (e.g. based on comparison to a reference model) as described above, and (c) optional consideration of the patient clinical condition and/or (d) optional demographic characteristics. The spine and elements are normalized as described above when necessary.

Assessment for abnormal conditions is typically based on benchmark model that optionally comprises norm margins and/or standard deviations in a given population, as described above (e.g. model 100). Optionally or additionally, the assessment refers to collection of norm margins and/or standard deviations in a given population separately from a spine model.

In some embodiments, the assessment and possible consequent diagnosis are performed or assisted according to decision making or classification algorithms, or combination thereof.

In some embodiments, the diagnosis is or can be carried out by a simple conditions based system or straightforward rule based system. For example, given a specific muscle, such as right multifidus muscle, deviation in cross sectional area above specific magnitude indicates that the source of pain may be weakness of multifidus muscle.

The assessment and/or diagnosis can be considered as a classification problem, For example, associating a given patient status in terms of elements characteristics with a diagnosis class). Therefore, some embodiments, the diagnosis is or can be carried out according to a classification algorithm or algorithms implemented in or as tools such as expert system, decision trees, neural networks, support vector machines, Gaussian Mixture Model system, radial basis function system, or statistical classification or analysis system.

In some embodiments, the assessment and/or diagnosis are augmented considering and/or implementing prior diagnosis or independent diagnosis (learning).

In some embodiments, the assessment may result in a single specific diagnosis or in several diagnosis possibilities. Optionally, diagnosis possibilities are prioritized according to given or determined probabilities (e.g. by learning).

It should be noted that many patients with low back pain do not exhibit acute condition of specific spine element. Rather, the patients exhibit sub-acute or subtle condition of several elements (deviations from a norm that individually are not considered severe) that cumulatively may cause instability or pain.

Some assessments are exemplified below.

### Spine Curvature Assessment

The spine curvature is being assessed against a reference model. For example, the curve is compared to a benchmark model. Since curves are dimensionless, the comparison can be a subtraction of both curves for each axial slice (Z axis), i.e. the distance between the curves axially. This distance is evaluated compared to the benchmark model standard deviation. If the curve deviates from the model at every point along the curve by more than determined factor of standard deviations the curve is designated as abnormal.

### Spine Curve and Muscles Deviations

Typically there is linkage between spine curve deviation (e.g. scoliosis, hyper-lordosis, kyphosis, hypo-lordosis, flat back) and stress on specific muscle groups. Certain deviation of the curve from the norm may increase the stress on specific muscle groups. For example, increase in the lumbar spine lordosis will increase the stress on the erector spinae muscles when exercising flexion and lifting movements, or, for example, Spine hypo-lordosis or flat back deviation along with weakness of the Erector Spinea muscle (or one or more of the following muscles Multifidus, Semispinalis thoracis, or Gluteus Maximus muscles) will cause instability, of different nature.

Although the deviation of the curve could be within the standard deviation of curves from the reference model curve, and muscles strength (weakness) could be within normal muscle strength, the combination of curve deviation together with muscle weakness could be cause for pain. Therefore, at least in some cases, deviation of the curve from the reference model curve is correlated with muscle characteristics as described above, for example, muscle cross sectional area or fat content.

### Transverse Processes Asymmetry

Below are some examples of measures or amounts used to assess vertebrae asymmetry, using as an example vertebra transverse processes.

In the description below reference is made to FIG. 11 that schematically illustrates neighbor vertebra bodies and transverse processes deviations, according to exemplary embodiments of the invention.

(i) Distances between two consecutive superior and inferior transverse processes apexes, shown as distances 1102a and 1102b at left and right sides, respectively.

(ii) Transverse processes apexes length, as defined by the distance between transverse processes apexes to vertebra body at the superior to inferior vertebrae and left and right sides, shown at the superior vertebra for left and right sides as 1104a and 1104b, respectively and at the inferior vertebra for left and right sides as 1104c and 1104d, respectively.

(iii) Angle between a line from transverse process apexes and a line between two vertebra bodies' centers at left and right sides, shown respectively as 1106a and 1106b.

(iv) Orientation of left and right transverse processes of the same vertebra, and top and bottom transverse process of consecutive vertebrae. Orientation is defined by the angle between the line from transverse process apexes to vertebra body center and the line between two vertebra bodies' centers 1110 at left and right sides shown respectively as 1108a and 1108b.

(v) Axial Orientation of left and right transverse processes of the same vertebra, and top and bottom transverse process of consecutive vertebrae. Orientation is the defined by the angle between a line from transverse process apexes to vertebra body center and the vertebra body midline (compare left to right, and superior to inferior vertebra).

With transverse processes asymmetry, spine morphology and biomechanics may be affected, depending on the type of asymmetry. For example differences in the length or the angle of the left and right transverse processes, may causes morphological changes such as different cross sectional areas of the left and right Psoas Major muscles, and different moment applied on the vertebra from Quadratus Lumborum muscles. Additionally, the processes asymmetry may eventually cause axial rotation of the vertebra, scoliosis, vertebra beveling, and muscle weakness. Differences in the left and right distances between two consecutive (superior and inferior) transverse processes apexes also may cause morphological and biomechanical instability due to changes in the spine curvature and different moment applied on the vertebra from Quadratus Lumborum muscles.

### Beveling of Several Vertebrae Deviations

Vertebra beveling as described above may affect spine morphology and biomechanics and may cause spine instability. Over a segment of the spinal cord, the beveling of individual vertebrae may be small and normal. However, cumulative beveling deviations with the same orientation can create spinal instability and impose stress on the spine. Cumulative beveling deviations are characterized, for example, by total beveling orientation (or total of beveling for several vertebrae) or other statistical or geometrical characteristics of beveling (e.g. average beveling, added curvature angle).

Common beveling deviations of several vertebrae should also be also correlated with deviations of the curve from a norm since they may increase the spine instability.

Beveling of several vertebrae deviations is typically classified as one of lateral plane beveling (scoliosis), anterior posterior plane beveling (usually kyphosis), or combination of both beveling planes.

### Nucleuses Orientations Over Several Vertebrae

Over a segment of the spinal cord, the orientations of individual discs relative to vertebra bodies over several vertebrae may be small and normal. However, cumulative nucleuses orientation deviations shared by several discs can create spinal instability and impose stress on the spine. Cumulative nucleuses orientation deviations are characterized, for example, by total orientation (or total relative intervertebral disc's nucleus center location for several intervertebral discs) or other statistical or geometrical characteristics of the relative intervertebral discs' nucleus center locations (e.g. average, curve, and curve distance from spine curve)

Common intervertebral disc's nucleus center location deviations of several intervertebral discs may also be also correlated with deviations of the curve from the norm since they may increase the spine instability.

Nucleuses relative locations over several vertebrae deviations are typically classified according to the deviation of the nucleuses as one of lateral plane deviation (the nucleuses are either to the left or right of the vertebra), anterior deviation (the nucleuses are located anterior to the vertebra), posterior deviation (the nucleuses are located posterior to the vertebra), or combination of both planes. Typically, lateral plane deviation increase muscles and ligaments stress, and anterior deviation typically causes more stress on the annulus in flexion and axial rotation, which consequently may cause tears and disc herniation. Posterior deviation may limit motion and may result in larger stress on the annulus in extension and axial rotation, which consequently may cause tears and disc herniation.

### Curve and Ligaments Deviations

Typically there is linkage between a spine curve and stress on ligaments as stabilizing elements. Certain deviations of the curve from a norm may increase the stress on specific ligaments. For example increase in the lumbar spine lordosis will increase the stress on the anterior ligament, posterior ligament, and ligamentum flavum.

Although the deviation of the curve could be within the standard deviation of curves from a benchmark model curve, and ligaments characteristic (e.g. density, cross sectional area) could be within normal ligaments values, the combination of curve deviation together with ligaments weakness could cause pain. Accordingly, deviation of the curve from the benchmark curve is correlated with ligaments characteristics as described above, for example, ligaments cross sectional area, or density.

A curve deviation type such as hyper-lordosis, kyphosis, hypo-lordosis, flat back or ligaments exhibiting weakness may indicate problematic spine configuration that, typically, should require specific exercise. For example, spine hyper-lordosis deviation along with deviation of the Ligamentum Flavuum may cause greater lumbar spine instability in flextion and extention movements, where spine hypo-lordosis or flat back deviation along with deviation of the posterior ligament may also cause instability.

### Muscle Asymmetry

Muscle asymmetry may cause spine instability and eventually development of low back pain. Training of one muscle, or muscle group, with neglecting to improve other muscles associated with movement together with the same muscle (for example as antagonist muscles) may create stress in these muscle that can cause pain. The stress may occur even if the muscles involved are all within norm or of better conditions (e.g. in term of size, density, fat content, or other physiological factors, as described above). Therefore, in addition to evaluation of muscles characteristics relative to benchmark model (or models healthy spine) and relative to spines of individuals having low back pain, other muscles that are working with muscles on various movements are evaluated as well. For example, left versus right side muscles characteristics, anterior versus posterior muscles characteristics, abdominal versus back muscles characteristics.

Muscle asymmetry deviations may also be also correlated with deviations of the curve from the norm since they may increase the spine instability. Consequently, specific asymmetry accompanied with certain curve deviation may increase the instability caused only by curve deviation or muscle asymmetry.

### Curvature Analysis Example

The non-limiting example below illustrates curvature analysis based on the description above.

A CT system was used to scan 21 individual with no spinal disorders and no history of LBP as reference group (healthy). The set of samples of spine curves were scaled to share a common superior point (at the level of T12 inferior endplate) and inferior point (at the level of L5 inferior endplate) and oriented along a common line (normalization). By sharing a common axis no registration between the curves was required, rendering the curves as dimensionless (patient independent). A benchmark model was formed in 3D as the median of location of all sample curves on cross sections along the curves along with standard deviation of location of all sample curves from the model curve. The model curve's spine curvature and torsion were calculates as the median curvature and torsion of all sample curves at each axial cut.

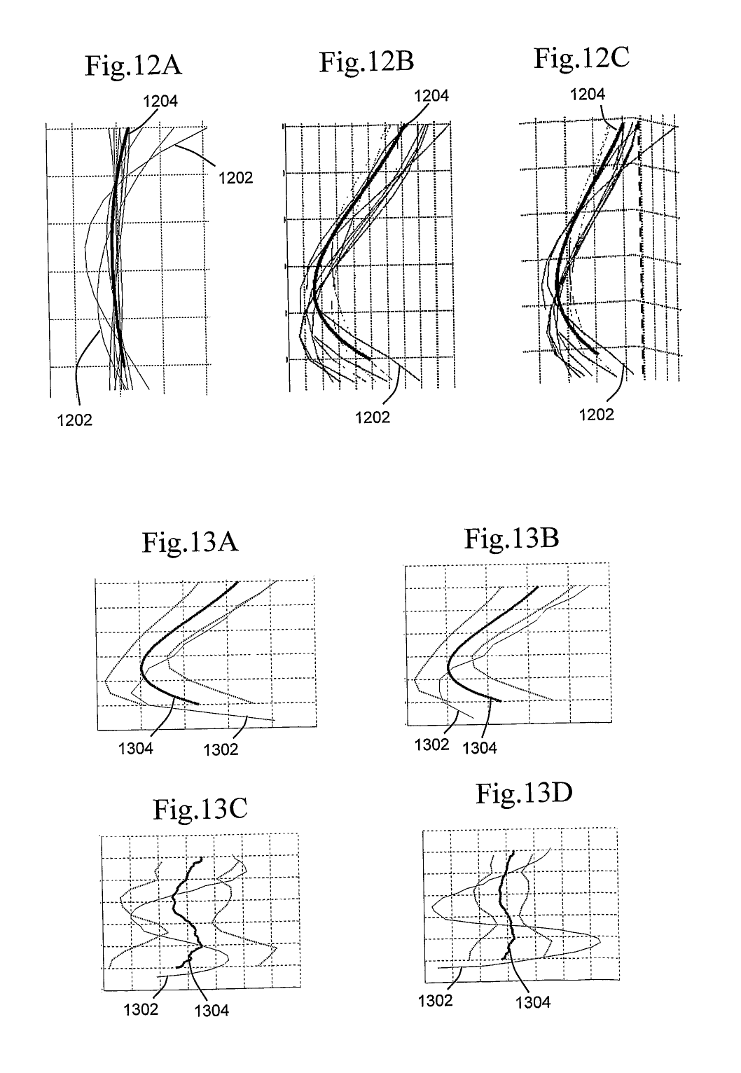

FIGS. 12A-C schematically illustrate three views of plurality of samples spine curves 1202 and a representative curve 1204 (as a benchmark curve) formed as a median of the plurality of curves.

A test group of 52 individuals was also scanned, where 16 had no history of back pain, 20 had previous LBP, 12 had non-specific LBP and 4 had scoliosis.

The spine's curvature of individuals of the test group is extracted from the CT studies and the curvatures are normalized to match the benchmark model of the reference group. The test curvatures were compared by evaluating distance measurement of the curvature and torsion relative to the model using distance measurement with respect to the standard deviation of the model. In case the distance deviation was larger than a set limit (3 standard deviations of the model as an example) the curve was denoted as abnormal.

### Exemplary Results

FIGS. 13A-D schematically illustrate test curves deviations 1302 of the test group from benchmark model curve 1304 in sagittal views (13A-B) and coronal views (13C-D), according to exemplary embodiments of the invention.

FIG. 13A illustrates mild hyper lordosis, FIG. 13B illustrates mild flat back,

FIG. 13C illustrates mild scoliosis and FIG. 13D illustrated scoliosis.

As FIGS. 13C-D illustrate, patients with scoliosis have large deviation from the benchmark model relative to patients with hyper lordosis (FIG. 13A) or flat back (FIG. 13B).

The results of the test group are exemplified in Table-3. The results were classified according to independent clinical assessment or diagnosis of the test group individuals (healthy, LBP history, current LBP and scoliotic) in order to identify relationships or correspondence with deviations from a benchmark model.

TABLE 3

| | | Patient Type | | | |
|---|---|---|---|---|---|
| | | | LBP | Current | |
| Measurement Type | | Healthy | History | LBP | Scoliosis |
| # of Patients | | 16 | 20 | 12 | 4 |
| Curve | Segments | 1 | 5 | 4 | 5 |
| | Percentage | 12.5% | 15% | 25% | 100% |
| | of patients | | | | |
| Curvature | Segments | 1 | 5 | 3 | 3 |
| | Percentage | 12.5% | 15% | 17% | 75% |
| | of patients | | | | |
| Torsion | Segments | 0 | 1 | 1 | 2 |
| | Percentage | 0% | 10% | 16% | 50% |
| | of patients | | | | |

Three types of deviations of the test groups were examined: (a) curve deviation from the benchmark model curve, (b) deviation of the curvatures along the curve, and (c) deviation of the torsion along the curve. For each examination type two values were recorded: (a) total amount of deviation segments per patient and (b) percentage of patients exhibiting deviations, wherein a segment denotes consecutive (connected) deviations (or single separate one).

The exemplary results demonstrate that spine curvature assessment method against a benchmark model according to embodiments of the present invention can be used to detect and quantify (at least approximately) pathologies such as scoliosis, lordosis or flat back. The exemplary results also demonstrate that patients with LBP have more deviations of the curve, curvature and torsion from the model than healthy patients. The unhealthy patients were not identified as having posture deformations and the deviations are sub-acute curvature deformation (i.e. deformations are not defined as pathologies) which may suggest that there is a correlation of posture problems and low back pain (chi-squared 0.01). Apparently there is a correlation between sub-acute curvature deformation (i.e. deformation that are not defined as pathologies) and low back pain, and therefore posture correction methods may aid LBP patients.

### Treatment Suggestions

Listed below are some examples of suggested possible and/or potential treatments or remedies which may be indicated (e.g. concluded or deduced) from the anatomical or morphological assessment, such as determined condition of a patient.

### Treatment Suggestions for Spine Curvature Assessment

Active (preventive) treatments for spine curvature deviations typically comprise posture correction training and muscles exercise. Muscle exercise is recommended for the muscles showing weakness, and/or secondary muscle supporting the weak muscles. Furthermore, since there is a strong connection between spine curve and stress on specific muscle groups, deviations of the curve from the norm may increase the stress on specific muscle groups. For example, hyper-lordosis (increase in the lumbar spine lordosis) typically increases the stress on the Erector Spinea muscles during flexion and lifting movements. Therefore specific curve deviations may also require exercise of certain muscle groups under increased strain relative to the norm.

Passive (preventive) treatments for spine curvature deviations typically comprise avoiding certain activities and movements (depending on the curve deviation type and muscle(s) showing weakness) or changes in ergonomics. For example, with hyper-lordosis avoiding activities such as weight lifting, breast-stroke swimming and movements such as flexion could be recommended whereas changing ergonomics such as using specifically designed chairs or hard mattress could also be recommended. In the case of hypo-lordosis avoiding activities such as cross country running or biking, driving motorcycle or movements such as extension could be recommended, whereas changing ergonomics such as using specifically designed chairs or soft mattress could also be recommended.

### Treatment Suggestions for Transverse Processes Asymmetry

Active (preventive) treatment in transverse processes asymmetry typically comprises using a back support belt or stabilizer or muscles exercise. For the example, due to the differences in the length of the left and right transverse processes, exercise that strengthens the muscles on the size that has short transverse process is recommended.

Passive (preventive) treatment in transverse processes asymmetry typically comprises avoiding certain activities and movements (depending on the curve deviation type and muscle(s) showing weakness) or changing ergonomics. For the example, due to the differences in the length of the left and right transverse processes, avoiding activities such as dancing (rotating the hips), movements such as axial rotation, and lateral flexion could be recommended, whereas changes in ergonomics such as using rotating chairs could also be recommended.

### Treatment Suggestions for Beveling of Several Vertebrae Deviations

Active (preventive) treatment in beveling of several vertebrae deviations typically comprises using a back support belt or stabilizer, posture correction training, or muscles exercise. For patients exhibiting lateral plane beveling (scoliosis), exercise of the muscles on the external side of the arc (created by the beveling) is recommended, along with posture correction training (e.g. exercise for scoliosis patients). For patients exhibiting anterior posterior plane beveling the treatment may depend on the type of curvature, typically hyper-kyphosis but hyper lordosis may also occur. For beveling suspected to lead to kyphosis the active treatment generally comprises posture correction training (e.g. exercise for patients having kyphosis). For patient with combination of both beveling the treatment typically comprises exercise of the muscles on the external side of the arc (created by the beveling), together with posture correction training.

Passive (preventive) treatment in beveling of several vertebrae deviations typically comprises avoiding certain activities and movements (depending on the deviation) or changes in ergonomics. For example, for patients exhibiting lateral plane beveling (scoliosis), avoiding activities such as participate in athletics without reservation (swimming and bicycling are typically better than running), movements such as hip flexion could be recommended, whereas changes in ergonomics that aid in correct posture could also be recommended.

### Treatment Suggestions for Nucleuses Orientations Over Several Vertebrae

Active (preventive) treatment for nucleuses orientations over several vertebrae generally comprises posture correction training or muscles exercise. For patients exhibiting lateral plane deviations, exercise of the back support muscles, and muscles involved with lateral flexion (Iliocostalis, Longissimus, Multifidus, External and internal Oblique, Quadratus Lumborum, Rhomboids, and Serraus Anterior) is typically recommended, along with posture correction training (e.g. exercise for scoliosis patients). For patients exhibiting anterior deviations exercise of the back support muscles and muscles involved with flexion (Rectus Abdominis, and Psoas Major), and axial rotation (Multifidus, Iliocostalis, Longissimus, External Oblique, and Splenius Thoracis) is typically recommended. For patients exhibiting posterior deviations exercise of the back support muscles and muscles involved with extention (Erector Spinea, Multifidus, and Semispinalis Thoracis) is typically recommended.

Passive (preventive) treatment for nucleuses orientations over several vertebrae generally comprises avoiding certain activities and movements (depending on the deviation) and changes in ergonomics. For patients exhibiting lateral plane deviations avoiding activities such as sleeping on side (should sleep on the back), participate in athletics without reservation (swimming and bicycling are better than running) or avoiding movements such as lateral flexion is typically recommended, whereas changes in ergonomics that ensure correct posture could also be recommended.

For patients exhibiting anterior deviations, avoiding activities such as heavy listing, participate in athletics without reservation (swimming and bicycling are better than running), movements such as flexion, or axial rotation is typically recommended, whereas changes in ergonomics that aid in correct posture could also be recommended. For patients exhibiting posterior deviations avoiding activities such as: heavy listing, participate in athletics without reservation (swimming and bicycling are better than running), movements such as extension, and axial rotation is typically recommended, whereas and changes in ergonomics that ensure correct posture could also be recommended.

### Treatment Suggestions for Curve and Ligaments Deviations

Active (preventive) treatment for curve and ligaments deviations comprises correction training or muscles exercise. Muscle exercise is typically recommended for muscles involved in movements controlled by the specific ligament assessed or diagnosed as having deviation. Furthermore, since typically there is a linkage between spine curve and stress on specific muscle groups, deviations of the curve from the norm may increase the stress on specific muscle groups. For example, hyper-lordosis (increase in the lumbar spine lordosis) may increase the stress on the erector spinea muscles in flexion and lifting movements and therefore specific curve deviations may also require exercise of certain muscle groups that have more strain.

Passive (preventive) treatment for curve and ligaments deviations comprises avoiding certain activities and movements (depending on the curve deviation type and muscle(s) showing weakness) or changes in ergonomics. For example, with hyper-lordosis avoiding activities such as weight lifting, breast-stroke swimming, and movements such as flexion is typically recommended whereas changes in ergonomics such as using specifically designed chairs or hard mattress. Hypo-lordosis calls for avoiding activities such as: cross country running or biking, driving motorcycle; avoiding movements such as extension, and changes in ergonomics such as using specifically designed chairs, and soft mattress could also be recommended.

### Treatment Suggestions for Muscle Asymmetry

Active (preventive) treatment in muscle asymmetry comprises posture correction training or muscles exercise. Muscle exercise is typically recommended for muscles showing weakness (when there is no muscle atrophy), and/or secondary muscle supporting the weak muscles.

Passive (preventive) treatment in muscle asymmetry comprises avoiding certain activities and movements (depending on the muscle or muscles showing weakness), or changes in ergonomics.

### Brief Elaboration on Muscles Segmentation

Following below is some exemplary non-limiting elaboration on a method for atlas-based automated segmentation of spine muscles, in addition to and continuation of an overview above. The description is presented here in order to avoid disrupting the order of the overview above.

### Affine Registration

The global transformation between spine case and the atlas is estimated by an affine transformation determined from correspondences between closely similar areas in both images using a block matching strategy where bones and body contour are used as a baseline for the correspondences. The scan is registered to the atlas using registration techniques on selected features or properties such as body surface and bone surfaces, such as mutual information or optical flow (on the scan volume) or Iterated Closest Point (ICP).

### Non-Rigid Registration

After affine registration of each spine case to the average shape atlas (AVG), non-rigid deformation is used to account for local differences between the spine case and the average atlas. Non-rigid deformation is modeled by a Free Form Deformation (FFD) based on B-splines. FFD employs normalized mutual information as a voxel-based similarity measure, since it is insensitive to intensity and contrast changes (e.g. bone and soft tissue density). Registration is achieved by minimizing a cost function, which represents a combination of the cost associated with the smoothness of the transformation and the cost associated with the image similarity. One can use a grid of control points defining a B-splines to determine the deformation. Each grid point is optimized individually to define local deformations. The B-splines are locally controlled making them computationally efficient even for a large number of control points. Furthermore, a multi-resolution approach of several hierarchical levels can be used to increase performance by decreasing the spacing between control points in consecutive levels.

### Tissue Classification

Subsequently to propagation of the probability atlas for segmentation of the muscle, a K-means tissue classifier is used to exclude any falsely included abdominal fat from the segmented muscle profile. The images assumed to contain several “tissue” classes (background, abdominal fat, muscles, bone, and abdominal viscera) and the intensity is classified using a standard K-means tissue classification algorithm.

### Atlas Creation and Propagation

AVG is generated iteratively where an arbitrary but “representative” case is selected as the initial reference case. In the first iteration each of the remaining images is registered to the selected reference case using an affine transformation. All the affinely registered cases and the reference case are then combined into an average (mean) atlas. Subsequent iterations involve all subjects including the reference case being registered to the average image by non-rigid transformation. Following each iteration, a new average image is generated and used as the input atlas for the subsequent iteration.

The probabilistic atlas (PA) for each muscle is generated (FIG. 2) by propagating the manual segmentations of this muscle for each case using the obtained affine and deformation field computed from the MR or CT into the atlas space. The resulting sets of segmentations were then combined into a probabilistic atlas.

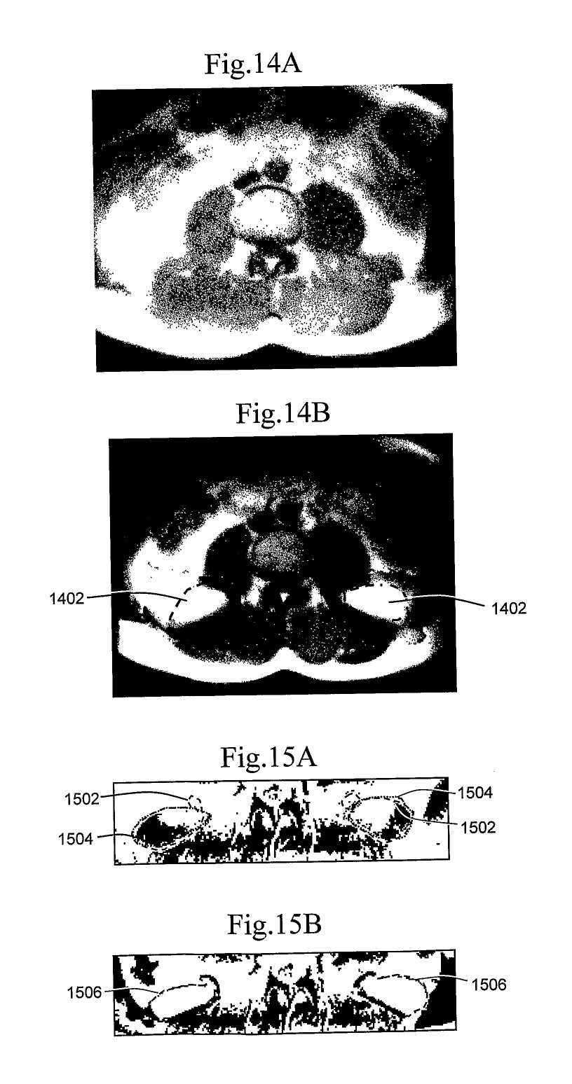

FIG. 14A illustrates an axial image (of CT or MRI or fusion thereof) view of an Average Shape Atlas of abdomen of an individual (converted to black and white), and FIG. 148 illustrates the image of FIG. 14A overlaid with probabilistic map of segmented Quadratus Lumborum muscle as indicated by dashed regions 1402, according to exemplary embodiments of the invention, using a total of 3 iterations.

### Segmentation Process

The acquired images of the patient by MRI or CT (or fusion thereof) are classified using a K-means algorithm. AVG atlas is registered to the patient case and the resulting registrations used to propagate probabilistic muscle to the patient's image space. Muscle segmentation is subsequently performed using geodesic active contours GAC or level sets. The level set implementation uses two inputs: (a) an initial input to seed the GAC level as the zero set representing the initial contour set based on the PA of the muscle and thresholded, for example, at 95%, and (b) the edge potential or speed image as the second input. The speed image is derived from the images which are then processed with anisotrophic diffusion and gradient and sigmoid functions in order to correspond to edges. The output level set generated by the GAC is then passed to a binary thresholding filter which produces a binary image representing the segmentation of the muscle.

FIG. 15A illustrates a partial view of the image of FIG. 14A (axial image of abdomen of an individual converted to black and white), with indications of manual 1502 and automatic 1504 segmentations, according to exemplary embodiments of the invention, showing the similarity of shape and size.

FIG. 15B illustrates a partial view of the image of FIG. 14A, with indications 1506 of probabilistic atlas, according to exemplary embodiments of the invention;

### System and Operation

In typical and preferred embodiments of the invention, the procedures and processes as described above are performed on a computer system (‘system’) comprising one or more processors and other equipment which may comprise or employ one or more processors. In some embodiments, the system use typical units and peripheral such as one or more of memory, buffers, input-output ports, monitors or input device. In some embodiments as least part of the system comprises a conventional personal computer or portable computer.

The system comprises and/or linked (such as by communication apparatus) to one or more programs, stored or wired in one or more media such as magnetic disc, SSD or flash drives, CD, ROM or others such as wired in FPGA or other ASIC devices. The program or programs implement algorithms such as described above, for example, one or more of image processing, feature extraction, classification, measurements, modeling, model comparison, assessment and/or diagnostics and one or more of input and output handling or presentation. Typically the one or more programs control and/or manage and/or coordinate activities of the system.

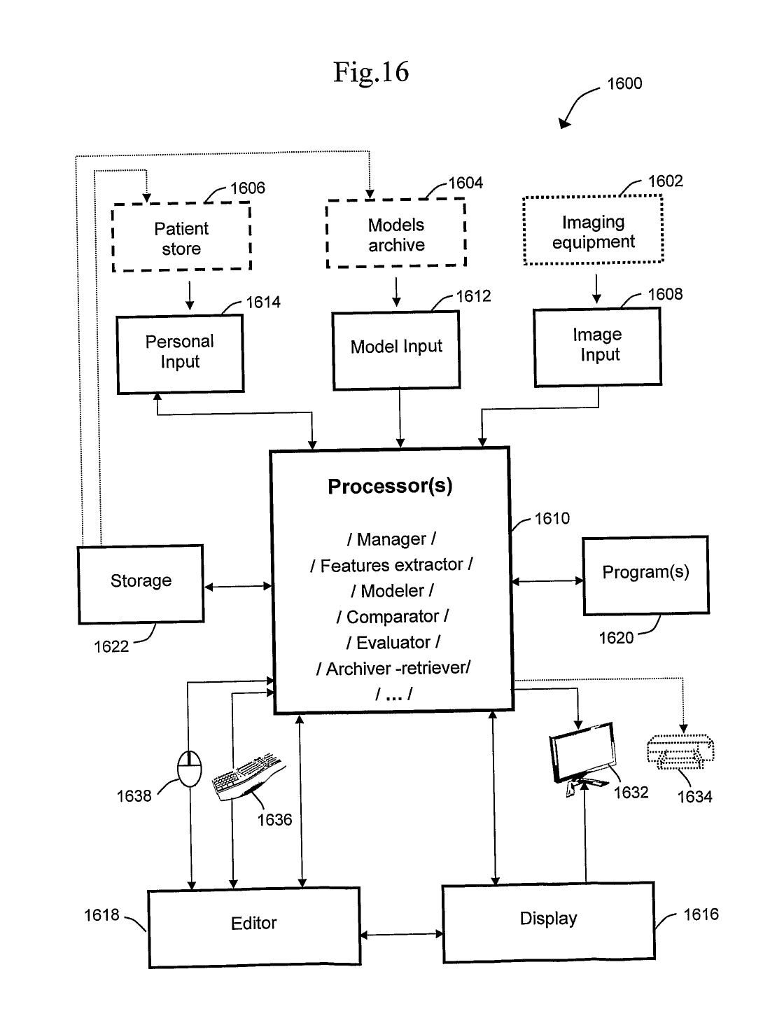

FIG. 16 schematically illustrates a system 1600 comprising components and functional units with at least partial relations illustrating data flow therebetween, according to exemplary embodiments of the invention. FIGS. 2A-F illustrating schematically data and actions for assessment of a patient's spine and respective description above are also referred to.

Imaging equipment 1602, comprising modalities such as CT, MRI or other modalities, is used to scan a patient and provide images of 3D data of the patients' spine. Imaging equipment 1602 is depicted but not necessarily comprised in system 1600. Image input 1608 receives images from imaging equipment 1602, optionally performing some initial preprocessing or organization of the images data, and provides the images data to processor 1610.

Model archive 1604 provides previously constructed models (e.g. model 100 of FIG. 1), using, for example, system 1600. Model input 1612 receives a model or models from model archive 1604, optionally performing some initial preprocessing or organization of the model elements, and provides the model to processor 1610.

Patient store 1606 provides information such as clinical data or demographic characteristics of the patient. Patient store 1606 optionally comprises two or more units or equipment, for example, PACS or other patient's records databases. In some embodiments at least part of the patient information is provide manually (via processor 1610 that optionally employs personal input 1614). Personal input 1614 receives patient data from patient store 1606, optionally performing some initial preprocessing or organization of the data, and provides the patient data to processor 1610. In some embodiments at least part of the patient information is provide manually (via processor 1610 that optionally employs personal input 1614).

In some cases model archive 1604 and patient store 1606 share a common storage or equipment or organization, at least partially.

Processor (or processors) 1610 works according to program (or programs) 1620 stored and/or wired (e.g. in FPGA) in or on one or more apparatus or devices. Programs 1620 implement tasks comprising one or more of (a) management and control of the operation of system 1600, (b) user interaction, (c) input and output, (d) peripherals handling, (d) storage and retrieval as well as one or more algorithms of such as (a) image processing, (b) anatomical and/or morphological feature extraction, (c) modeling a spine, (d) comparing spines, (e) evaluating spine anatomy or morphology or (f) generation diagnosis of a patient's spine.

System 1600 comprises a display unit 1616, operated by processor 1610, providing presentations of data such as images (e.g. CT 3D studies), spine elements (e.g. vertebrae, discs, spinal cord), properties of elements (e.g. density of spinal cord, fat contents of muscle), geometrical relations between or within a part (or between parts of two models) or diagnosis. Values are optionally displayed as alphanumeric (textual) values and/or symbols representing magnitudes or relative magnitudes (e.g. arrow length).

The presentation may be organized or formed according intended person or operators (see some more below) and provides capabilities and/or tools for manipulating the displayed objects (e.g. spine or parts thereof) such a by scroll, zoom, pan, windowing (range of shades), contrast, brightness, sharpening, and any other display, imaging or image processing operations.

System 1600 comprises an editor unit 1618, operated by processor 1610, providing capabilities such as organizing or modifying, adding or deleting data objects. For example, applying image processing operations on images, adjusting measurements locations or results, removing erroneous entities or artifacts. Optionally editor unit 1618 is used to accept patient's data manually, possibly employing personal input 1614.

System 1600 comprises storage unit 1622, operated by processor 1610, handing storage and retrieval of data objects and optionally exporting data to external units not comprised in System 1600. For example, when a patient's model is constructed the model stored in storage unit 1622 which, consequently exports it to model archive 1604, or as a diagnosis is completed (and optionally accepted by a clinician) storage unit 1622 exports the diagnosis to patient store. In some embodiments, storage unit 1622 is functional unit using memory devices (e.g. RAM) or in some embodiments storage unit 1622 comprises, at least partially memory device or devices.

In some embodiments, models stored in or on storage unit 1622, or optionally exported to model archive 1604, may be retrieved later for review or comparison with a current or more up to date spine model. Likewise, in some embodiments, a diagnosis stored in or on storage unit 1622, or optionally exported to personal store 1606, may be retrieved later for comparison or consideration in generating a diagnosis (‘learning’).

System 1600 comprises one or more presentation peripherals, non-limitedly represented by monitor 1632 and optional printer 1634. System 1600 comprises one or more input peripherals, non-limitedly represented by keyboard 1636 and mouse 1638.

It should be emphasized that one or more of functional units such as image input 1608, model input 1612, personal input 1614, display unit 1616, editor 1618 and possibly storage unit 1622 are included or comprised, at least partially, in processor 1610 and optionally programs 1620, or in equipment comprising processor 1610 and optionally programs 1620.

It should also be noted that memory (e.g. as RAM, ROM or other data storage devices) is implicitly comprised and used in system 1600 and is not particularly shown in FIG. 16.

In some embodiments of the invention, system 1600 operates in one or more operational modes and optionally sub-modes, possibly one or more of the sub-modes operating concurrently (e.g. tasks, threads, or processes). For example, input of one patient image or data is carried out while another patient is diagnosed or her data are edited.

Display unit 1616 of system 1600 enables to present (display, show) patient specific diagnosis and demonstrating the pathologies to the patient in a suitable fashion, aiding the operator (e.g. orthopedist) to determine the condition and/or diagnosis of the patient's spine.

In some embodiments of the invention, system 1600 with display unit 1616 has at least three presentation (viewing) modes of operation: (i) diagnosis mode for clinicians (e.g. radiologists, or orthopedic doctor), (ii) treatment mode therapist (e.g. physiotherapists, health instructors), and (iii) patient mode patient understanding and involvement.

### Diagnosis Mode

Diagnosis mode is designed for clinicians (e.g. an orthopedic physician or surgeon) for determining an assessment or diagnosis. As such, the diagnosis mode allows extensive interaction of the operator (e.g. a clinician) with the system relative to other modes.

In the diagnosis mode, all or most of the identified elements and features are presented, using methods of the art or custom methods, for example, volume rendering, multi planar reformation (MPR), tabular forms, diagrams or charts.

In some embodiments, elements are presented (displayed) with characteristics or properties thereof. Optionally the presentation is graphical, such as a line showing a disk height with the height value as an annotation, or an element or property with a contour showing a respective now maximal value, and optionally the presentation is textual such as a list or table of characteristics or values.

In some embodiments, a combined view of a patient's spine image and a spine model (e.g. benchmark model) is presented. For example, presenting the model elements on or in the image portraying the correlations or deviations of the imaged parts relative to the model, or presenting the image and model side by side (or at least partly overlaying) while maintaining correlation and prelateship between patient's image parts and the model (e.g. by annotations or connecting lines).

In some embodiments, the patient's spine image and/or model thereof and/or benchmark model are presented with alignment of corresponding elements such as by side by side presentation or overlay or partial overlay or solid display of parts of one model overlaid with contour display of another model, optionally with different colors or shades representing the match or deviation between corresponding parts. Optionally or additionally, the deviation is presented textually or as a symbol.

In some embodiments, the patient's spine image and/or model thereof and/or benchmark model are presented with characteristics (properties, metrics) of elements portrayed either as individual values of as in deviations from a reference, such as by portraying a magnitude as numerical values and/or in color or symbol code. For example, bone density is portrayed by dark red or yellow for reduced density to white for healthy bone, or a distance is portrayed by a line length of a circle diameter, optionally or additionally with further color coding for additional dimension or aspect (e.g. size for magnitude and color for deviation).

In some embodiments of diagnosis mode, features or elements or properties can be edited, such as by using editor unit 1618. For example, correction of segmentation, adding missing feature such as a point, curve, or surface or changing numerical values. In some embodiments, such as by using editor unit 1618 and/or display unit 1616, image processing operations may be invoked and applied on the patient image such as on selected or identified elements or areas and/or measurements are applied on the image of elements.

In some embodiments of diagnosis mode, based on the extracted features or model a visually realistic (or approximation) view of the patient's spine is presented such as by using display unit 1616. For example, by using openGL with or without graphic acceleration hardware, or Virtual Reality Modeling Language (VRML) or Scalable Vector Graphics (SVG). In some embodiments, the view is edited such as by editor unit 1618, where areas of interest or components of elements or features are modified, added or removed, optionally based on reference to diagnosis or patient data and/or condition (e.g. illness).

In some embodiments a component or element is selected, such as by editor unit 1618, and one or more measurements are determined, usually generated or aided by system 1600. Typically an assessment or diagnosis is determined according to the on one or more measurements based on the selected elements. Optionally, an assessment or diagnosis are determined or generated based on or with reference to pervious measurements and/or diagnosis, possibly providing more elaborate measurements or assessments than typically performed.

In some embodiments a component or element is selected, such as by editor unit 1618, and measurement and/or diagnosis are determined or provided by the operator (e.g. clinician) with respect or reference to the selected object. Optionally, a measurement and/or diagnosis are generated or aided by system 1600 with respect or reference to the selected object.

In some embodiments of diagnosis mode, a diagnosis is presented, such as by using display unit 1616, in a tabular (e.g. alphanumeric) and/or graphical format. In some embodiments, the diagnosis may be edited such by editor unit 1618, modifying the diagnosis such as by external information (e.g. from other modalities such as ultrasound).

In some embodiments, the diagnosis may be annotated and/or classified such by editor unit 1618, providing comments or acceptance or confirmation of the diagnosis.

In some embodiments of the invention, based on presentation of the diagnosis mode, presentations suitable for other modes (e.g. treatment or patient modes) are defined or formed, such as by using display unit 1616 and/or editor unit 1618.

### Treatment Mode

In typical embodiments of the invention, the editing capabilities are limited or disabled relative to the diagnosis mode, optionally also with limited variants of presentation.

### Patient Mode

In typical embodiments of the invention, the editing capabilities are disabled relative to the diagnosis or treatment modes, optionally also with limited variants of presentation, providing diagnosis, model view and obtaining diagnosis or information for selected elements of objects.

### Advantages

Possible or probable advantages of the invention relative to the current state comprise one or more of the following.

(i) Increase the specific diagnosis of patients.

(ii) Increase the reliability diagnosis.

(iii) Indication of patient specific treatment plan.

(iv) Fast and easy diagnosis.

(v) Offer patient encouragement through involvement in the treatment.

### General

All trademarks are the property of their respective owners.

The following non-limiting characterizations of terms are applicable in the specification and claim unless otherwise specified or indicated in or evidently implied by the context, and wherein a term denotes also variations, derivatives, inflections and conjugates thereof.

The terms ‘processor’ or ‘computer’ (or system thereof) is used herein as ordinary context of the art, typically comprising additional elements such memory or communication ports. Optionally or additionally, terms ‘processor’ or ‘computer’denote any deterministic apparatus capable to carry out a provided or an incorporated program and/or access and/or control data storage apparatus and/or other apparatus such as input and output ports. The terms ‘processor’ or ‘computer’ denote also a plurality of processors or computers connected, and/or linked and/or otherwise communicating, possibly sharing one or more other resources such as memory.

The terms ‘software’ and ‘program’ may be used interchangeably, and denote one or more instructions or directives or circuitry for performing a sequence of operations that generally represent an algorithm and/or other process or method. The program is stored in or on a medium (e.g. RAM, ROM, disc, etc.) accessible and executable by an apparatus such as a processor or other circuitry.

The processor and program may constitute the same apparatus, at least partially, such as an array of electronic gates (e.g. FPGA, ASIC) designed to perform a programmed sequence of operations, optionally comprising or linked with a processor or other circuitry.

In case electrical or electronic equipments is disclosed it is assumed that an appropriate power supply is used for the system operation.

The terms ‘about’, ‘close’, ‘approximate’, ‘practically’ and ‘comparable’ denote a respective relation or measure or amount or quantity or degree yielding an effect that has no adverse consequence or effect relative to the referenced term or embodiment or operation or the scope of the invention.

The terms ‘substantial’, ‘considerable’, ‘significant’, ‘appreciable’ (or synonyms thereof) denotes with respect to the context a measure or extent or amount or degree which encompass most or whole of a referenced entity, or is sufficiently large or close or effective or important relative to a referenced entity or with respect the referenced subject matter.

The terms ‘negligible’, ‘slight’ and ‘insignificant’ (or synonyms thereof) denote, a sufficiently small respective relation or measure or amount or quantity or degree to have practical consequences relative to the referenced term and on the scope of the invention.

The terms ‘similar’, ‘resemble’, ‘like’ and the suffix ‘-like’ denote shapes and/or structures and/or operations that look or proceed as, or approximately as the referenced object.

The terms ‘constant’, ‘uniform’, ‘continuous’, ‘simultaneous’, ‘equal’ and other seemingly definite terms denote also close or approximate respective terms.

The terms ‘vertical’, ‘perpendicular’, ‘parallel’, ‘opposite’, ‘straight’ and other angular and geometrical relationships denote also approximate yet functional and/or practical, respective relationships.

The teens ‘usually, ‘preferred’, ‘preferably’, ‘typical’ or ‘typically’ do not limit the scope of the invention or embodiments thereof.

The terms ‘comprises’, ‘comprising’, ‘includes’, ‘including’, ‘having’ and their inflections and conjugates denote ‘including but not limited to’.

The term ‘may’ denotes an option which is either or not included and/or used and/or implemented, yet the option constitutes at least a part of the invention.

Unless the context indicates otherwise, referring to an object in the singular form (e.g. ‘a thing” or “the thing”) does not preclude the plural form (e.g. “the things”).

The present invention has been described using descriptions of embodiments thereof that are provided by way of example and are not intended to limit the scope of the invention or to preclude other embodiments. The described embodiments comprise various features, not all of which are necessarily required in all embodiments of the invention. Some embodiments of the invention utilize only some of the features or possible combinations of the features. Alternatively and additionally, portions of the invention described or depicted as a single unit may reside in two or more separate entities that act in concert or otherwise to perform the described or depicted function. Alternatively and additionally, portions of the invention described or depicted as two or more separate physical entities may be integrated into a single entity to perform the described/depicted function. Variations related to one or more embodiments may be combined in all possible combinations with other embodiments.

When a range of values is recited, it is merely for convenience or brevity and includes all the possible sub-ranges as well as individual numerical values within that range. Any numeric value, unless otherwise specified, includes also practical close values enabling an embodiment or a method, and integral values do not exclude fractional values. A sub-range values and practical close values should be considered as specifically disclosed values.

In the specifications and claims, unless particularly specified otherwise, when operations or actions or steps are recited in some order, the order may be varied in any practical manner.

Terms in the claims that follow should be interpreted, without limiting, as characterized or described in the specification.

1-47. (canceled)

48. A method for assessment of a medical condition of a patient's spine, comprising:
constructing a first computerized model representing the anatomy of the spine wherein the constructing is based on extraction of one or more anatomical elements of the spine from at least one image of the spine; and
determining a medical condition of the spine based on a comparison between the computerized model and reference anatomical values.

49. The method according to claim 48, wherein the reference anatomical values comprise normal values derived from clinical data.

50. The method according to claim 49, wherein the normal values are demographically-matched to the patient.

51. The method according to claim 48, wherein the reference anatomical values comprise values of a second computerized model representing the anatomy of a benchmark spine;
wherein the second computerized model is constructed based on extraction of one or more anatomical elements of at least one image of each of a plurality of reference healthy spines.

52. The method according to claim 51, wherein the reference healthy spines are selected to match at least one of demographic characteristic, clinical status and clinical information of the patient.

53. The method according to claim 48, wherein representing the anatomy of the spine comprises representing one or more of quantitative, morphological and geometrical properties of the spine.

54. The method according to claim 48, wherein representing the anatomy of the spine comprises representing quantitative geometrical relationships between and within the elements.

55. The method according to claim 48, wherein representing the anatomy of the spine comprises representing a hierarchy between the elements.

56. The method according to claim 48, wherein the at least one image is acquired from at least one imaging apparatus selected from the group consisting of: CT, MRI, PET, US and X-ray.

57. A method for aiding an assessment of a spine, comprising:
(a) providing a model representing the anatomy of a test spine; and
(b) displaying a presentation of at least a part of the model of the test spine in a manner adapted for determining an assessment of the spine by an operator.

58. The method according to claim 57, wherein the presentation comprises a graphical presentation, a textual presentation of at least one property relating to the at least a part of the spine, a depiction of the relationship of the at least one part to a respective norm, or combinations thereof.

59. The method according to claim 57, further comprising measuring at least one property of the at least a part of the test spine.

60. The method according to claim 57, further comprising modifying a property of the at least a part of the test spine.

61. The method according to claim 57, further comprising modifying a graphical representation of the at least a part of the test spine.

62. The method according to claim 57, further comprising:
(a) providing a model representing the anatomy of a reference spine; and
(b) displaying a presentation of at least a part of the model of the test spine with a presentation of at least a corresponding part of the model of the reference spine wherein the presentation is adapted to portray a difference between the corresponding parts.

63. The method according to claim 62, wherein the presentation comprises a depiction of the relationship difference between the corresponding parts to a respective norm.

64. The method according to claim 62, further comprising measuring the difference between the corresponding parts.

65. The method according to claim 57, wherein the presentation is carried out by at least one computer according to at least one program comprised in a storage device.

66. A non-transitory computer readable storage medium having instructions stored therein, which, when executed by a computer, cause the computer to:
construct a computerized model representing the anatomy of a spine, wherein the constructing is based on extraction of one or more anatomical elements of the spine from at least one image of the spine; and
determine a medical condition of the spine based on a comparison between the computerized model and reference anatomical values.

67. The non-transitory computer readable storage medium according to claim 66, further comprising instructions stored therein, which, when executed by the computer, cause the computer to perform one or more of:
(a) extract one or more anatomical elements of a spine from an image,
(b) construct a model representing the anatomy of a spine using extracted anatomical elements,
(c) determine a condition of a spine based on a model of a spine,
(d) compare a model of the spine to another model of another spine,
(e) display a presentation of at least a part of a model of a spine in a manner adapted for determining an assessment of the spine by an operator,
(f) display a presentation of at least a part of a model of a spine with a presentation of at least a corresponding part of another model of another spine wherein the presentation is adapted to portray a difference between the corresponding parts.
